(19) United States
(12) Patent Application Publication
(10) Pub. No.: US 2026/0002150 A1
(43) Pub. Date: Jan. 1, 2026

(54) TETRAZINE-DERIVED LINKERS FOR SINGLE GUIDE RNAS

(71) Applicant: UNIVERSITY OF MASSACHUSETTS, Westborough, MA (US)

(72) Inventors: Jonathan K. Watts, Worcester, MA (US); Phillip D. Zamore, Northborough, MA (US); Amena Arif, Worcester, MA (US); Zexiang Chen, Worcester, MA (US); Gitali Devi, Westborough, MA (US); Erik J. Sontheimer, Auburndale, MA (US)

(21) Appl. No.: 18/852,289

(22) PCT Filed: Mar. 29, 2023
(86) PCT No.: PCT/US2023/016735

§ 371 Date: Sep. 27, 2024

Related U.S. Application Data
(60) Provisional application No. 63/324,925, filed on Mar. 29, 2022.

(51) Int. Cl. C12N 15/11; C12N 9/22; C12N 15/90

(52) U.S. Cl. CPC C12N 15/11; C12N 9/226; C12N 15/907; C12N 2310/20; C12N 2310/351

(57) ABSTRACT

CRISPR-Cas genome editing technology has applications in biomedical research and therapeutics that has led to an increased demand for guide RNAs. Synthesis of chemically modified single-guide RNAs (sgRNAs)>100 nt remains a bottleneck. A tetrazine ligation method is disclosed herein for the preparation of sgRNAs that overcomes this synthesis challenge. For example, a tetrazine moiety on a 3′-end of crRNA and a norbornene moiety on a 5′-end of tracrRNA permits ligation between crRNA and tracrRNA to form an sgRNA under mild conditions. Tetrazine-ligated sgRNAs allow efficient genome editing as demonstrated by reporter models and endogenous loci in human cells. A structural modification of the linker moiety permits achievement of high efficiency editing.

### CROSS-REFERENCE TO RELATED APPLICATIONS

The present application claims the benefit of U.S. Provisional Patent Application No. 63/324,925, filed on Mar. 29, 2022, which is incorporated herein by reference.

### THE STATEMENT OF GOVERNMENTAL SUPPORT

This invention was made with government support under grant no. TR002668 awarded by the National Institutes of Health. The Government has certain rights in the invention.

### FIELD OF THE INVENTION

The present invention is related to the field of genome editing. In particular, a tetrazine-derived linker is disclosed to assemble a CRISPR sgRNA. sgRNA tetrazine-derived linkers are shown to have improved gene editing efficiencies in a manner based upon linker length and structural modification. The presently disclosed tetrazine-derived linkers are ligated between a crRNA and a tracrRNA as an alternative to conventional sgRNA tetraloop linker synthesis methods.

### BACKGROUND

CRISPR-Cas technology has revolutionized genome editing. Its broad and fast-growing application in biomedical research and therapeutics has led to increased demand for guide RNAs. However, synthesis of chemically modified single-guide RNAs (sgRNAs)>100 nt remains a bottleneck.

Most CRISPR-Cas systems, including S. pyogenes Cas9 (Spy-Cas9), utilize single-guide RNAs (sgRNAs) of 100+ nucleotides. Oligonucleotides this long are expensive to synthesize, and yields tend to be low. A dual-guide approach can be used, consisting of two short RNA pieces (crRNA and tracrRNA) assembled by hybridization. However, sgRNA is more effective than dual-guide RNA for genome editing. Finn et al., “A Single Administration of CRISPR/Cas9 Lipid Nanoparticles Achieves Robust and Persistent In Vivo Genome Editing” Cell Rep 22 (9): 2227-2235 (2018) [1]. Therefore, efficient scalable production of safe and effective sgRNAs would be expected to improve the art.

What is needed in the art are safe and effective sgRNAs that are greater than 100 nt comprising chemical linkers between the crRNA and tracrRNA.

### SUMMARY OF THE INVENTION

The present invention is related to the field of genetic engineering. In particular, a tetrazine-derived linker is disclosed for a CRISPR sgRNA. sgRNA tetrazine linker complexes are shown to have improved synthetic accessibility and high gene editing efficiencies in a manner based upon linker length and structural modification. The presently disclosed tetrazine-derived linkers are ligated between a crRNA and a tracrRNA as an alternative to the conventional sgRNA tetraloop linker.

In one embodiment, the present invention contemplates a single guide ribonucleic acid (sgRNA) comprising a crRNA domain, a tracrRNA domain and a linker comprising a tetrazine-derived moiety and a dienophile-derived moiety. In one embodiment, the dienophile-derived moiety comprises a norbornene-conjugated nucleic acid. In one embodiment, the dienophile-derived moiety comprises a bicyclo[6.1.0] nonyne-conjugated nucleic acid. In one embodiment, the dienophile-derived moiety comprises a trans-cyclooctene-conjugated nucleic acid. In one embodiment, the linker is ligated between said crRNA molecule and said tracrRNA molecule. In one embodiment, the tetrazine-derived moiety is ligated to a 3′ terminus of the crRNA. In one embodiment, the dienophile-derived moiety is ligated to a 5′ terminus of the tracrRNA. In one embodiment, the tetrazine-derived moiety is ligated to a 5′ terminus of the tracrRNA. In one embodiment, the norbornene-derived moiety is ligated to a 3′ terminus of the crRNA. In one embodiment, the linker molecule further comprises an octaethylene glycol (PEG8) molecule. In one embodiment, the linker molecule further comprises a tetraethylene glycol (PEG4) molecule. In one embodiment, the crRNA molecule further comprises at least three additional nucleotides. In one embodiment, the tracrRNA molecule further comprises at least three additional nucleotides. In one embodiment, the tetrazine-derived moiety further comprises a pyridazine. In one embodiment, the linker molecule includes, but is not limited to, tetrazine-derived long linker 2, tetrazine-derived long linker 3, tetrazine-derived long linker 4, tetrazine-derived long linker 5 and tetrazine-derived long linker 6. In one embodiment, the sgRNA is at least 100 nt in length.

In one embodiment, the present invention contemplates a method, comprising: a) providing; i) a tracrRNA domain comprising a 5′-alkene or 5′-alkyne dienophile moiety; ii) a crRNA domain comprising a 3′-tetrazine moiety; and b) ligating the 5-alkene or 5′-alkyne dienophile moiety and 3′-tetrazine to create an sgRNA comprising a linker molecule having a dienophile-derived moiety and a tetrazine-derived moiety. In one embodiment, the sgRNA is at least 90 nt in length. In one embodiment, the 5′-alkene or 5′-alkyne dienophile moiety is derived from a 5′-norbornene phosphoramidite. In one embodiment, the 5′-alkene or 5′-alkyne dienophile is derived from a 5′-bicyclo[6.1.0] nonyne phosphoramidite. In one embodiment, the 5′-alkene or 5′-alkyne phosphoramidite is derived from a 5′-trans-cyclooctene phosphoramidite. In one embodiment, the ligating comprises an incubation at room temperature. In one embodiment, the incubation is approximately twenty hours. In one embodiment, the ligating further comprises a buffer consisting of Tris-HCl and NaCl. In one embodiment, the 3′-tetrazine moiety is derived from an NHS ester. In one embodiment, the 3′-tetrazine moiety further comprises an oligoethylene glycol or polyethylene glycol moiety. In one embodiment, the polyethylene glycol molecule is octaethylene glycol (PEG8). In one embodiment, the polyethylene glycol molecule is tetraethylene glycol (PEG4). In one embodiment, the method is metal-free. In one embodiment, the method is copper-free.

In one embodiment, the present invention contemplates a method, comprising: a) providing: i) a single guide ribonucleic acid (sgRNA) comprising a crRNA domain, a tracrRNA domain and a linker comprising a tetrazine-derived moiety and a dienophile-derived moiety; ii) a Cas9 nuclease; and iii) a target nucleic acid that is at least partially complementary to said sgRNA; b) contacting said sgRNA with said Cas9 nuclease to create a Cas9 nuclease/sgRNA complex; and c) hybridizing said Cas9 nuclease/sgRNA complex to said target nucleic acid, wherein the sequence of said target nucleic acid is edited. In one embodiment, the Cas9 nuclease is an inactivated Cas9 nuclease (dCas9). In one embodiment, the Cas9 nuclease is a Cas9 nickase (nCas9). In one embodiment, the target nucleic acid is derived from a gene. In one embodiment, the gene includes, but is not limited to, CCR5, HEK3, TRAC, and HPRT. In one embodiment, the target nucleic acid is linked to a genetic disease or disorder. In one embodiment, the edited target nucleic acid corrects said genetic disease or disorder. In one embodiment, the linker molecule is ligated between said crRNA molecule and said tracrRNA molecule. In one embodiment, the tetrazine-derived moiety is ligated to a 3′-terminus of the crRNA molecule. In one embodiment, the norbornene moiety is ligated to a 5′-terminus of the tracrRNA molecule. In one embodiment, the tetrazine-derived moiety is ligated to a 5′-terminus of the crRNA molecule. In one embodiment, the norbornene moiety is ligated to a 3′-terminus of the tracrRNA molecule. In one embodiment, the linker further comprises octaethylene glycol (PEG8). In one embodiment, the linker further comprises tetraethylene glycol 4 (PEG4). In one embodiment, the crRNA domain further comprises at least three additional nucleotides. In one embodiment, the tracrRNA domain further comprises at least three additional nucleotides. In one embodiment, the tetrazine-derived moiety further comprises a pyridazine. In one embodiment, the linker includes, but is not limited to, a tetrazine-derived long linker 2, a tetrazine-derived long linker 3, a tetrazine-derived long linker 4, a tetrazine-derived long linker 5 and a tetrazine-derived long linker 6. In one embodiment, the sgRNA is at least 100nt in length.

In one embodiment, the present invention contemplates a single guide (sg) ribonucleic acid (RNA) comprising a crRNA domain, a tracrRNA domain and a linker region comprising a tetrazine moiety and a norbornene moiety. In one embodiment, the linker region is between the crRNA domain and the tracrRNA domain. In one embodiment, the tetrazine moiety is ligated to a 3′ crRNA end. In one embodiment, the norbornene moiety is ligated to a 5′ tracrRNA end. In one embodiment, the tetrazine moiety is ligated to a 5′ tracrRNA end. In one embodiment, the norbornene moiety is ligated to a 3′ crRNA end. In one embodiment, the linker region further comprises PEG8. In one embodiment, the linker region further comprises PEG4. In one embodiment, the crRNA domain is extended by at least three nucleotides. In one embodiment, the tracrRNA domain is extended by at least three nucleotides. In one embodiment, the tetrazine-derived moiety comprises a pyridazine. In one embodiment, the linker region includes, but is not limited to, a tetrazine long linker 2, a tetrazine long linker 3, a tetrazine long linker 4, a tetrazine long linker 5 and a tetrazine long linker 6. In one embodiment, the norbornene moiety is replaced with a dienophile. In one embodiment, the dienophile includes, but is not limited to, a bicyclo[6.10] nonane, a trans-cyclooctene, or another unsaturated group suitable for undergoing a cycloaddition reaction with tetrazine.

In one embodiment, the present invention contemplates a method, comprising: a) providing: i) a single guide (sg) ribonucleic acid (RNA) comprising a crRNA domain, a tracrRNA domain and a linker region comprising a tetrazine-derived moiety and a norbornene-derived moiety; ii) a Cas9 nuclease; and iii) a target nucleic acid that is at least partially complementary to the sgRNA tetrazine linker complex; b) contacting the sgRNA with the Cas9 nuclease to create a Cas9 nuclease/sgRNA complex; and c) hybridizing the Cas9 nuclease/sgRNA complex, wherein the sequence of the target nucleic acid is edited. In one embodiment, the target nucleic acid is derived from a gene. In one embodiment, the gene includes, but is not limited to, CCR5, HEK3, TRAC, and HPRT. In one embodiment, the target nucleic acid is in a cell used for biological or biomedical research. In one embodiment, the target nucleic acid is linked to a genetic disease or disorder. In one embodiment, the edited target nucleic acid treats the genetic disease or disorder. In one embodiment, the target nucleic acid is in a patient. In one embodiment, the target nucleic acid is in a cell designed to be reintroduced into a patient. In one embodiment, the linker region is ligated between the crRNA molecule and the tracrRNA molecule. In one embodiment, the tetrazine moiety is ligated to a 3′ crRNA end. In one embodiment, the norbornene moiety is ligated to a 5′ tracrRNA end. In one embodiment, the tetrazine moiety is ligated to a 5′ crRNA end. In one embodiment, the norbornene moiety is ligated to a 3′ tracrRNA end. In one embodiment, the linker region further comprises PEG8. In one embodiment, the linker region further comprises PEG4. In one embodiment, the crRNA region is extended by at least three nucleotides. In one embodiment, the tracrRNA region is extended by at least three nucleotides. In one embodiment, the tetrazine moiety comprises a pyridazine. In one embodiment, the linker region includes, but is not limited to, a tetrazine long linker 2, a tetrazine long linker 3, a tetrazine long linker 4, a tetrazine long linker 5 and a tetrazine long linker 6.

In one embodiment, the present invention contemplates a method, comprising: a) providing: i) a single guide ribonucleic acid (sgRNA) comprising a crRNA domain, a tracrRNA domain and a linker comprising a tetrazine-derived moiety and an alkene dienophile-derived moiety; ii) a Cas9 protein with one nuclease domain inactivated (nCas9) or both nuclease domains inactivated (dCas9) appended to a base editor domain; and iii) a target nucleic acid that is at least partially complementary to said sgRNA; b) contacting said sgRNA with said Cas9 protein to create a Cas9 protein/sgRNA complex; and c) hybridizing said Cas9 protein/sgRNA complex to said target nucleic acid, wherein the sequence of said target nucleic acid is edited. In one embodiment, the present invention contemplates a method, comprising: a) providing: i) a single guide ribonucleic acid (sgRNA) comprising a crRNA domain, a tracrRNA domain and a linker comprising a tetrazine-derived moiety and an alkene dienophile-derived moiety; ii) a Cas9 protein with one nuclease domain inactivated (nCas9) or both nuclease domains inactivated (dCas9) appended to an epigenetic modification domain; and iii) a target nucleic acid that is at least partially complementary to said sgRNA; b) contacting said sgRNA with said Cas9 protein to create a Cas9 protein/sgRNA complex; and c) hybridizing said Cas9 protein/sgRNA complex to said target nucleic acid, wherein the epigenetic status of said target nucleic acid is modified.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−10% of a given measurement.

The term “substitute for” as used herein, refers to the switching the administration of a first compound or drug to a subject for a second compound or drug to the subject.

As used herein, the term “CRISPR” or “Clustered Regularly Interspaced Short Palindromic Repeats” refers to an acronym for a genome editing system originally discovered by its association with DNA loci that contain multiple, short, direct repetitions of base sequences. Each repetition contains a series of bases followed by 30 or so base pairs known as “spacer” sequence. The spacers are short segments of DNA from a virus or other invasive nucleic acid, and may serve as a ‘memory’ of past exposures to facilitate an adaptive defense against future invasions. Doudna et al. Genome editing. The new frontier of genome engineering with CRISPR-Cas9″ Science 346 (6213): 1258096 (2014) [2].

As used herein, the term “Cas” or “CRISPR-associated (cas)” refers to genes often associated with CRISPR repeat-spacer arrays.

As used herein, the term “Cas9” refers to any nuclease derived from type II CRISPR systems. A wild type Cas9 enzyme generates double-strand breaks in DNA, with two active cutting sites (the HNH and RuvC domains), one for each strand of the double helix.

As used herein, the term “catalytically active Cas9” refers to an unmodified Cas9 nuclease (e.g., wild type) comprising full nuclease activity.

As used herein, the term “nickase Cas9” or “nCas9” refers to a Cas9 nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact. Jinek et al., “A programmable dual-RNA-guided DNA endonuclease in adaptive bacterial immunity” Science 337 (6096): 816-821 (2012) [3] and Cong et al. Multiplex genome engineering using CRISPR/Cas systems” Science 339 (6121): 819-823 (2013) [4].

The term “single-guide RNA” or “sgRNA” refers to a combination of tracrRNA and spacer RNA into a single molecule that, mixed with Cas9, could find and cleave DNA targets through Watson-Crick pairing between the guide sequence within the sgRNA and the target DNA sequence, Jinek et al. A programmable dual-RNA-guided DNA endonuclease in adaptive bacterial immunity” Science 337 (6096): 816-821 (2012) [3].

The term, “trans-activating crRNA”, “tracrRNA” as used herein, refers to a small trans-encoded RNA. There are several pathways of CRISPR activation, one of which requires a tracrRNA, which plays a role in the maturation of crRNA. TracrRNA is complementary to the repeat sequence of the pre-crRNA, forming an RNA duplex. This is cleaved by RNase III, an RNA-specific ribonuclease, to form a crRNA/tracrRNA complex via duplex formation. This complex acts as a guide for the endonuclease Cas9, which cleaves the invading nucleic acid.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

The terms “protospacer adjacent motif recognition domain”, “PAM Interacting Domain” or “PID” as used herein, refers to a Cas9 amino acid sequence that comprises a binding site to a DNA target PAM sequence.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence of nucleic acids. Furthermore, the molecular arrangement may comprise a lipid bilayer or other biological material.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337 (6096): 816-821 (2012) [3] Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage. Esvelt et al., “Orthogonal Cas9 proteins for RNA-guided gene regulation and editing” Nat Methods 10 (11): 1116-1121 (2013) [5]. For example, this would allow one Cas9 isoform (e.g. S. pyogenes Cas9 or SpyCas9) to function as a nuclease programmed by a sgRNA that may be specific to it, and another Cas9 isoform (e.g. N. meningitidis Cas9 or NmeCas9) to operate as a nuclease-dead Cas9 that provides DNA targeting to a binding site through its PAM specificity and orthogonal sgRNA. Other Cas9s include S. aureus Cas9 or SauCas9 and A. naeslundii Cas9 or AnaCas9.

The term “truncated” as used herein, when used in reference to either a polynucleotide sequence or an amino acid sequence means that at least a portion of the wild type sequence may be absent. In some cases, truncated guide sequences within the sgRNA or crRNA may improve the editing precision of Cas9. Fu, et al. “Improving CRISPR-Cas nuclease specificity using truncated guide RNAs” Nat Biotechnol. 2014 March;32 (3): 279-284 (2014) [6].

The term “base pairs” as used herein, refer to H-bonded pairs of specific nucleobases (also termed nitrogenous bases), that are the building blocks of nucleotide sequences that form a primary structure of both DNA and RNA. Double-stranded DNA may be characterized by specific hydrogen bonding patterns. Base pairs may include, but are not limited to, guanine-cytosine and adenine-thymine base pairs.

The term “specific genomic target” as used herein, refers to any pre-determined nucleotide sequence capable of binding to a single site within the genome of interest, after association with a Cas9 protein as contemplated herein. The target may include, but may be not limited to, a nucleotide sequence complementary to a programmable DNA binding domain, a nucleotide sequence complementary to a single guide RNA, a protospacer adjacent motif recognition sequence, an on-target binding sequence and an off-target binding sequence.

As used herein, the term “edit,” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” or ‘linked to” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The term “portion” when used in reference to a nucleotide sequence refers to fragments of that nucleotide sequence. The fragments may range in size from 5 nucleotide residues to the entire nucleotide sequence minus one nucleic acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency.

This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity. An oligonucleotide sequence which is a “homolog” is defined herein as an oligonucleotide sequence which exhibits greater than or equal to 50% identity to a sequence, when sequences having a length of 100 bp or larger are compared.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G: C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, “a nucleic acid sequence” even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences”. Upon transcription into pre-mRNA, a gene typically contains exons and introns; introns are then removed or “spliced out” from the pre-mRNA and are absent from the mature messenger RNA (mRNA) transcript. Introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript.

These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

### BRIEF DESCRIPTION OF THE FIGURES

FIG. 1A-B presents exemplary mechanisms regarding tetrazine ligation chemistry for RNA ligation.

FIG. 1A: General mechanism of tetrazine-derived ligation.

FIG. 1B: A tetrazine ligation strategy to create an sgRNA molecule.

FIG. 2 presents exemplary data showing a ³¹P NMR analysis for norbornene-methanol phosphoramidite.

FIG. 3A-C presents exemplary data of comparing sgRNAs with a tetrazine short linker 1 and sgRNAs with a tetrazine long linker 2.

FIG. 3A: A representative illustration of an sgRNA with a tetrazine short linker 1 and an sgRNA with a tetrazine long linker 2.

FIG. 3B: A representative dose titration of an sgRNA with a tetrazine short linker 1 and an sgRNA with a tetrazine long linker 2 in an HEK-293T TLR1 assay. Cas9: sgRNA ratio is fixed at 1:3.

FIG. 3C: A representative Cas9: sgRNA ratio titration of an sgRNA with a tetrazine short linker 1 and an sgRNA with a tetrazine long linker 2 in an HEK-293T TLR1 assay. RNP dosage is fixed at 2.5 pmol. Mean±s.d. of 3 independent biological replicates.

FIG. 4 presents exemplary data of tetrazine ligation reaction of TLR1 sgRNA with linker 1 or linker 2.

FIG. 5 presents exemplary data showing denaturing PAGE for tetrazine ligation of TLR1 sgRNA with linker 1 or linker 2.

FIGS. 6A-C presents exemplary HPLC-MS data for TLR1-linker 1 (no PS) sgRNA.

FIG. 6A: HPLC chromatogram.

FIG. 6B: Deconvoluted MS. sgRNA peak region is indicated by red rectangle. The lower-mass peak represents an apparent doubly charged ion (m/2z) due to imperfections in the deconvolution.

FIG. 6C: Zoom-in of sgRNA peaks in deconvoluted MS. sgRNA peak indicated by red arrow. Other peaks are likely cation (NH4+, Na+, K+, etc.) adducts.

FIGS. 7A-C presents exemplary HPLC-MS data for TLR1-linker 2 (no PS) sgRNA.

FIG. 7A: HPLC chromatogram.

FIG. 7B: Deconvoluted MS. sgRNA peak region is indicated by red rectangle.

The lower-mass peak represents an apparent doubly charged ion (m/2z) due to imperfections in the deconvolution.

FIG. 7C: Zoom-in of sgRNA peaks in deconvoluted MS. sgRNA peak indicated by red arrow. Other peaks are likely cation (NH4+, Na+, K+, etc.) adducts.

FIGS. 8A-C presents exemplary HPLC-MS data for TLR1-linker 2 (with PS) sgRNA.

FIG. 8A: HPLC chromatogram.

FIG. 8B: Deconvoluted MS. sgRNA peak region is indicated by red rectangle.

FIG. 8C: Zoom-in of sgRNA peaks in deconvoluted MS. sgRNA peak indicated by red arrow. Other peaks are likely cation (NH4+, Na+, K+, etc.) adducts.

FIGS. 9A-C presents exemplary HPLC-MS data for CCR5-Linker 2 sgRNA.

FIG. 9A: HPLC chromatogram.

FIG. 9B: Deconvoluted MS. sgRNA peak region is indicated by red rectangle.

FIG. 9C: Zoom-in of sgRNA peaks in deconvoluted MS. sgRNA peak indicated by red arrow. Other peaks are likely cation (NH4+, Na+, K+, etc.) adducts.

FIGS. 10A-C presents exemplary HPLC-MS data for HEK3-Linker 2 sgRNA.

FIG. 10A: HPLC chromatogram.

FIG. 10B: Deconvoluted MS. sgRNA peak region is indicated by red rectangle.

FIG. S10C: Zoom-in of sgRNA peaks in deconvoluted MS. sgRNA peak indicated by red arrow. Other peaks are likely cation (NH4+, Na+, K+, etc.) adducts.

FIGS. 11A-C presents exemplary HPLC-MS data for TRAC-Linker 2 sgRNA.

FIG. 11A: HPLC chromatogram.

FIG. 11B: Deconvoluted MS. sgRNA peak region is indicated by red rectangle.

FIG. 11C: Zoom-in of sgRNA peaks in deconvoluted MS. sgRNA peak indicated by red arrow. Other peaks are likely cation (NH4+, Na+, K+, etc.) adducts.

FIGS. 12A-C presents exemplary HPLC-MS data for HPRT-Linker 2 sgRNA.

FIG. 12A: HPLC chromatogram,

FIG. 12B: Deconvoluted MS. sgRNA peak region is indicated by red rectangle.

FIG. 12C: Zoom-in of sgRNA peaks in deconvoluted MS. sgRNA peak indicated by red arrow. Other peaks are likely cation (NH4+, Na+, K+, etc.) adducts.

FIG. 13A-D presents exemplary data comparing genome editing efficiency between sgRNAs with a tetrazine long linker 2 and sgRNAs with a tetraloop linker.

FIG. 13A: Titration of sgRNAs with a tetrazine long linker 2 and sgRNAs with a tetraloop linker in the HEK-293T TLR1 assay, with Cas9: sgRNA ratio fixed at 1:3.

FIG. 13B: Cas9: sgRNA ratio titration of sgRNAs with a tetrazine long linker 2 and sgRNAs with a tetraloop linker in the HEK-293T TLR1 assay, with RNP dosage fixed at 2.5 pmol.

FIG. 13C: Dose titration of sgRNAs with a tetrazine long linker 2 and sgRNAs with a tetraloop linker for endogenous loci (e.g., CCR5, HEK3, TRAC, and HPRT) in HEK-293T cells, with Cas9: sgRNA ratio fixed at 1:3.

FIG. 13D: Cas9: sgRNA ratio titration of sgRNAs with a tetrazine long linker 2 and sgRNAs with a tetraloop linker for endogenous loci (e.g., CCR5, HEK3, TRAC, and HPRT) in HEK-293T cells, with RNP dose fixed at 2.5 pmol. Mean±s.d. of 3 independent biological replicates

FIG. 14A-B presents exemplary designs and data of standard and different linker ligated sgRNAs.

FIG. 14A: Embodiments of various sgRNA linker designs: i) standard tetraloop linker: ii) tetrazine long linker 2; iii) tetrazine long linker 3; iv) tetrazine long linker 4; v) tetrazine long linker 4, vi) tetrazine long linker 5; and vii) tetrazine long linker 6.

FIG. 14B: Comparison of HPRT sgRNA genome editing efficiency between sgRNAs with a tetraloop linker and sgRNAs with tetrazine linkers 2, 3, 4, 5, and 6 at 2.5 pmol and 5 pmol RNP dosages in HEK-293T cells. Mean #s.d. of 3 independent biological replicates.

FIG. 15A-C presents exemplary HPLC-MS data for HPRT-Linker 3 sgRNA.

FIG. 15A: HPLC chromatogram.

FIG. 15B: Deconvoluted MS. sgRNA peak region is indicated by red rectangle.

FIG. 15C: Zoom-in of sgRNA peaks in deconvoluted MS. sgRNA peak indicated by red arrow. Other peaks are likely cation (NH4+, Na+, K+, etc.) adducts.

FIGS. 16A-C presents exemplary HPLC-MS data for HPRT-Linker 4 sgRNA.

FIG. 16A: HPLC chromatogram.

FIG. 16B: Deconvoluted MS. sgRNA peak region is indicated by red rectangle.

FIG. 16C: Zoom-in of sgRNA peaks in deconvoluted MS. sgRNA peak indicated by red arrow. Other peaks are likely cation (NH4+, Na+, K+, etc.) adducts.

FIGS. 17A-C presents exemplary data HPLC-MS for HPRT-Linker 5 sgRNA.

FIG. 17A: HPLC chromatogram.

FIG. 17B: Deconvoluted MS. sgRNA peak region is indicated by red rectangle.

FIG. 17C: Zoom-in of sgRNA peaks in deconvoluted MS. sgRNA peak indicated by red arrow. Other peaks are likely cation (NH4+, Na+, K+, etc.) adducts.

FIGS. 18A-C presents exemplary HPLC-MS data for HPRT-Linker 6 sgRNA.

FIG. 18A: HPLC chromatogram.

FIG. 18B: Deconvoluted MS. sgRNA peak region is indicated by red rectangle.

FIG. 18C: Zoom-in of sgRNA peaks in deconvoluted MS. sgRNA peak indicated by red arrow. Other peaks are likely cation (NH4+, Na+, K+, etc.) adducts.

FIG. 19 presents exemplary data showing a comparison of linker 2-and linker 5-ligated sgRNAs with stem-length-matched sgRNA and dgRNA controls at TLR1, HPRT, CCR5 and TRAC loci in HEK-293T cells. Mean+s.d. of 3 biological replicates.

FIG. 20 presents exemplary electrophoretic mobility shift assay (EMSA) of seven TLR1 gRNAs used in FIG. 19.

FIG. 21 presents a chemical structure of 3′-phthalimide (PT)-amino-modifier C3 CPG and norbornene-methanol phosphoramidite.

FIG. 22 presents a 3 P NMR analysis for norbornene-methanol phosphoramidite.

FIG. 23 presents a chemical structure of methyltetrazine-NHS ester and methyltetrazine-PEG8-NHS ester.

FIG. 24 presents representative Sanger sequencing files for indel analysis by ICE3 HEK3 Locus.

FIGS. 25A-C presents exemplary HPLC-MS data for TLR1-Linker 5 sgRNA.

FIG. 25A: HPLC chromatogram.

FIG. 25B: Deconvoluted MS. sgRNA peak region is indicated by red rectangle.

FIG. 25C: Zoom-in of sgRNA peaks in deconvoluted MS. sgRNA peak indicated by red arrow. Other peaks are likely cation (NH4+, Na+, K+, etc.) adducts.

FIGS. 26A-C presents exemplary HPLC-MS data for CCR5-Linker 5 sgRNA.

FIG. 26A: HPLC chromatogram.

FIG. 26B: Deconvoluted MS. sgRNA peak region is indicated by red rectangle.

FIG. 26C: Zoom-in of sgRNA peaks in deconvoluted MS. sgRNA peak indicated by red arrow. Other peaks are likely cation (NH4+, Nat, K+, etc.) adducts.

FIGS. 27A-C presents exemplary HPLC-MS data for TRAC-Linker 5 sgRNA.

FIG. 27A: HPLC chromatogram.

FIG. 27B: Deconvoluted MS. sgRNA peak region is indicated by red rectangle.

FIG. 27C: Zoom-in of sgRNA peaks in deconvoluted MS. sgRNA peak indicated by red arrow. Other peaks are likely cation (NH4+, Na+, K+, etc.) adducts.

FIGS. 28A-C presents exemplary data of flow cytometry gating.

FIG. 28A; Live

FIG. 28B: Single cells.

FIG. 28C: mCherry

### DETAILED DESCRIPTION OF THE INVENTION

The present invention is related to the field of genetic engineering. In particular, a tetrazine-derived linker is disclosed for a CRISPR sgRNA. sgRNA tetrazine-derived linkers are shown to have improved gene editing efficiencies in a manner based upon linker length and structural modification. The presently disclosed tetrazine-derived linkers are ligated between a crRNA and a tracrRNA as an alternative to the conventional sgRNA tetraloop linker.

In one embodiment, the present invention contemplates a tetrazine ligation method for the preparation of sgRNAs. A tetrazine moiety on the 3′ or 5′-end of a crRNA and a norbornene moiety on the 5′ or 3′-end of a tracrRNA enables successful ligation between crRNA and tracrRNA to form sgRNA under mild conditions. Tetrazine-ligated sgRNAs allow efficient genome editing of reporter and endogenous loci in human cells. High efficiency gene editing can be achieved with structural modification of a tetrazine-derived linker.

The data presented herein describes an sgRNA comprising a tetrazine-derived linker that supports efficient gene editing activity, equivalent to and/or better than a conventional sgRNA comprising a tetraloop linker. Moreover, the presently disclosed tetrazine ligation strategy is easy to implement and scale, because a long, invariant tracrRNA can be chemically synthesized in bulk quantity, ready to be ligated to the shorter, sequence-variable crRNA designed to hybridize with a desired target sequence. Therefore, sgRNAs comprising tetrazine-derived linkers provide an attractive alternative for efficient CRISPR genome editing. The data suggest that the production of tetrazine-derived linkers for other Cas nucleases that have even longer guide RNAs than Cas9 can expand the utility of CRISPR genome editing in biomedical research and therapeutics development. Edraki et al., “A Compact, High-Accuracy Cas9 with a Dinucleotide PAM for In Vivo Genome Editing” Molecular Cell 73 (4): 714-726 (2019) [7].

### I. CRISPR Cas9 Technology

CRISPR-Cas genome editing has advanced biomedical research and has been suggested as a potential therapeutic modality. Urnov F. D., “Genome Editing B.C. (Before CRISPR): Lasting Lessons from the “Old Testament.”” The CRISPR Journal 1 (1): 34-46 (2018) [8]. CRISPR-Cas systems have been reported to use programmable guide RNAs that direct sequence-specific DNA cleavage by Cas nucleases. Jinek et al., “A Programmable Dual-RNA-Guided DNA Endo-nuclease in Adaptive Bacterial Immunity” Science 337 (6096): 816-821 (2012) [3]; Jinek et al., “RNA-Programmed Genome Editing in Human Cells” eLife 2: e00471 (2013) [9]; Cong et al., “Multiplex Genome Engineering Using CRISPR/Cas Systems” Science 339 (6121): 819-823 (2013) [4]; and Mali et al., “RNA-Guided Human Genome Engineering via Cas9” Science 339 (6121): 823-826 (2013) [10].

CRISPR-mediated editing has been performed in cells or organisms by DNA, RNA, or RNP-based delivery of the effector and guide RNA. Glass et al., “Engineering the Delivery System for CRISPR-Based Genome Editing” Trends in Biotechnology 36 (2): 173-185 (2018) [11]. Direct chemical synthesis can be used to generate homogenous chemically modified gRNAs with an improved efficiency, enhanced stability, reduced off-target editing, and improved delivery and cellular uptake. Mir et al., “Heavily and Fully Modified RNAs Guide Efficient SpyCas9-Mediated Genome Editing” Nature Communications 9 (1): 1-9 (2018) [12]; Yin et al., “Structure-Guided Chemical Modification of Guide RNA Enables Potent Non-Viral in Vivo Genome Editing” Nature Biotechnology 35 (12): 1179-1187 (2017) [13]; Rahdar et al., “Synthetic CRISPR RNA-Cas9-Guided Genome Editing in Human Cells” Proc. Natl. Acad. Sci. U.S.A. 112 (51): E7110-7117 (2015) [14]; Cromwell et al., “Incorporation of Bridged Nucleic Acids into CRISPR RNAs Improves Cas9 Endonuclease Specificity” Nature Communications 9 (1): 1-11 (2018) [15]; Yin et al., “Partial DNA-Guided Cas9 Enables Genome Editing with Reduced off-Target Activity” Nature Chemical Biology 14 (3): 311-316 (2018) [16]; Rueda et al., “Mapping the Sugar Dependency for Rational Generation of a DNA-RNA Hybrid-Guided Cas9 Endonuclease” Nature Communications 8 (1): 1-11 (2017) [17]; Finn et al., “A Single Administration of CRISPR/Cas9 Lipid Nanoparticles Achieves Robust and Persistent In Vivo Genome Editing” Cell Rep 22 (9): 2227-2235 (2018) [1]; Hendel et al., “Chemically Modified Guide RNAs Enhance CRISPR-Cas Genome Editing in Human Primary Cells” Nature Biotechnology 33 (9): 985-989 (2015) [18]; Ryan et al., “Improving CRISPR-Cas Specificity with Chemical Modifications in Single-Guide RNAs” Nucleic Acids Res 46 (2): 792-803 (2018) [19]; and Basila et al., “Minimal 2′-O-Methyl Phosphorothioate Linkage Modification Pattern of Synthetic Guide RNAs for Increased Stability and Efficient CRISPR-Cas9 Gene Editing Avoiding Cellular Toxicity” PLOS ONE 12 (11): e0188593 (2017) [20].

### II. Conventional Single Guide RNA Synthesis Techniques

Ligation of short synthetic RNAs has been reported to offer an alternative to synthesizing a long RNA. However, enzymatic ligation is time-consuming and difficult to scale. El-Sagheer et al., “New Strategy for the Synthesis of Chemically Modified RNA Constructs Exemplified by Hairpin and Hammerhead Ribozymes” PNAS 107 (35): 15329-15334 (2010) [21]. Instead, chemical ligation was found to be relatively easy to implement and scale.

CRISPR-Cas genome editing has profoundly advanced biomedical research and holds promise as a therapeutic modality [8]. CRISPR-Cas systems use programmable guide RNAs that direct sequence-specific DNA cleavage by Cas nucleases [3, 4, 9, 10]. CRISPR-mediated gene editing can be performed in cells or organisms by DNA, RNA, or RNP-based delivery of the effector and guide RNA [11]. Direct chemical synthesis has been used to generate chemically modified gRNAs with improved efficiency, enhanced stability, reduced off-target editing, and improved delivery and cellular uptake relative to unmodified guides [1, 12-20].

Most CRISPR-Cas systems, including S. pyogenes Cas9 (SpyCas9), use single-guide RNAs (sgRNAs) of 100+ nucleotides [3]. Oligonucleotides this long are expensive to synthesize, and yields tend to be low. A dual-guide approach has been reported, consisting of two short RNA pieces (crRNA and tracrRNA) assembled by hybridization [3, 12]. However, sgRNAs are more effective than dual-guide RNAs for genome editing in many cases. For example, after lipid nanoparticle delivery of Cas9 mRNA and guide RNA to the mouse liver, sgRNA provided substantially higher in vivo editing than dual-guide RNA [1]. Therefore, an efficient scalable synthesis of safe and effective sgRNAs remains a challenge.

Tetraloop sgRNA Linker

Standard sgRNA designs comprise a crRNA fused to a tracrRNA by a 4-nt (GAAA) linker. Jinek et al., “A Programmable Dual-RNA-Guided DNA Endo-nuclease in Adaptive Bacterial Immunity” Science 337 (6096): 816-821 (2012) [3]. This linker forms a tetraloop that protrudes from the nuclease in CRISPR-Cas9 structures and it has been suggested that SpyCas9 can accommodate the structural changes induced by the tetraloop. Nishimasu et al., “Crystal Structure of Cas9 in with Guide RNA and Target DNA” Cell 156 (5): 935-949 (2014) [22]; and Jiang et al., “CRISPR-Cas9 Structures and Mechanisms” Annu. Rev. Biophys. 46 (1): 505-529 (2017) [23]; Jinek et al., “Structures of Cas9 Endonucleases Reveal RNA-Mediated Conformational Activation” Science 343 (6176) (2014) [24].

B. Copper-Based sgRNA Ligation

Ligation of short synthetic RNAs offer an alternative to an end-to-end synthesis of a long gRNA (e.g., >100 nts). Conventional enzymatic ligation, however, is time-consuming and difficult to scale [21]. Instead, the present invention contemplates a chemical ligation which is relatively easy to implement and scale.

For example, copper-catalyzed azide-alkyne cycloaddition (CuAAC) chemistry has recently been used to ligate two RNA components into a sgRNA. However, these ligated sgRNAs were significantly less effective than in vitro transcribed sgRNAs, although their efficiency can be aided by installing chemical modifications on the ligated sgRNAs. Taemaitree et al., “An Artificial Triazole Backbone Linkage Provides a Split-and-Click Strategy to Bioactive Chemically Modified CRISPR SgRNA” Nat Commun 10 (1): 1610 (2019) [25]. Moreover, the copper catalyst is toxic, and therefore products must be carefully purified before in vivo use. Copper can also have side reactions with phosphorothioate (PS) linkages which are desired for stability and uptake of sgRNAs in vivo. Gaetke L., “Copper Toxicity, Oxidative Stress, and Antioxidant Nutrients” Toxicology 189 (1-2): 147-163 (2003) [26]; Jewett et al., “Cu-Free Click Cycloaddition Reactions in Chemical Biology” Chem. Soc. Rev. 39 (4): 1272 (2010) [27]; and Neumann et al., “The CuAAC: Principles, Homogeneous and Heterogeneous Catalysts, and Novel Developments and Applications” Macromol. Rapid Commun. 41 (1): 1900359 (2020) [28].

Copper-free SPAAC reactions also ligated sgRNAs that were functional for in vitro DNA cleavage, but cellular data was not collected. Taemaitree et al., “An Artificial Triazole Backbone Linkage Provides a Split-and-Click Strategy to Bioactive Chemically Modified CRISPR SgRNA” Nat Commun 10 (1): 1610 (2019) [25]. Moreover, copper catalysts are known to be toxic, and therefore products synthesized with copper must be carefully purified before in vivo administration [26-28]. Copper has also been reported to cause side reactions with phosphorothioate (PS) linkages which contribute to stability and uptake of sgRNAs in vivo. Therefore, metal-free chemical ligation cell-free methods were tested to prepare sgRNAs [25]. Taemaitree et al also demonstrated that a copper-free SPAAC reaction could ligate sgRNAs that were functional for in vitro DNA cleavage, but did not present cellular data.

III. sgRNAs Comprising Tetrazine-Derived Linkers

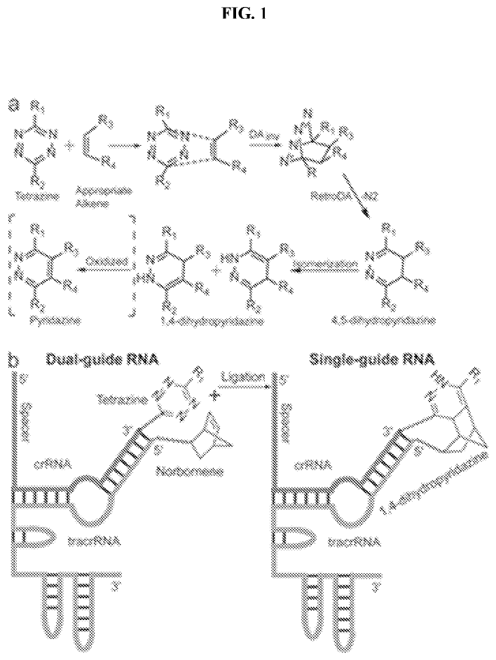

In one embodiment, the present invention contemplates a method comprising a nucleotide-free sgRNA linker. In one embodiment, the synthesis method comprises a copper-free chemical ligation of a crRNA and a tracrRNA to synthesize an sgRNA. In one embodiment, the sgRNA comprises a tetrazine-derived linker between a 3′-end of crRNA and a 5′-end of tracrRNA. See, FIG. 1B.

Although it is not necessary to understand the mechanism of an invention, it is believed that sgRNAs comprising tetrazine-derived linker molecules can form functional complexes with any type of Cas9 nuclease. For example, a catalytically active Cas9 nuclease, a catalytically inactive Cas9 nuclease (dCas9) or a Cas9 nickase nuclease (nCas9). Alternatively, other forms of Cas9 nucleases are also contemplated to function with sgRNAs comprising a tetrazine-derived linker molecule such as base editors, CRISPR-based activators and CRISPR-based repressors.

Base editors are constructs comprising a Cas9 nuclease appended to a deaminase protein. For example, the deaminase protein may be a cytidine deaminase that converts cytosine to uracil in the target DNA strand. There are a large number of different cytidine deaminases that have been used in cytosine base editors-natural deaminases, such as rAPOBEC1, and engineered variants such as BE4. The type of cytidine deaminase domain can be swapped within cytosine base editors to change the base conversion efficiency in different sequence contexts. Alternatively, the deaminase may be an adenine deaminase that converts adenine to inosine in the target DNA strand. There are a number of different adenine deaminases that have been evolved for use in adenine base editors, such as TadA7.10 and TadA8e. The type of adenine deaminase domain can be swapped within adenine base editors to change the base conversion efficiency in different sequence contexts. Huang, et. al. Nat Protoc. 16 (2): 1089-1128 (2021) [29].

CRIPSR-based activators and repressors create epigenetic modifications. Epigenome editing is a tool in which the DNA or histone is modified at specific sites in the genome using engineered molecules. This strategy requires precise targeting which is accomplished through the use of nuclease-deficient Cas9 (dCas9). However, unlike genome editing, epigenome editing does not affect genome DNA sequence. It has been reported that a Cas9 repressor may be based on a C-terminal fusion of a rationally designed bipartite repressor domain, KRAB-MeCP2, to nuclease-dead Cas9. Yeo et al., “An enhanced CRISPR repressor for targeted mammalian gene regulation” Nature Methods 15, pages 611-616 (2018) [30]. A dCas9-p300 CRISPR gene activator system has been reported that is based on a fusion of dCas9 to the catalytic histone acetyltransferase (HAT) core domain of the human E1A-associated protein p300. Hilton et al., “Epigenome editing by a CRISPR-Cas9-based acetyltransferase activates genes from promoters and enhancers” Nat Biotechnol. 33 (5): 510-517 (2015) [31]; and Ji et al., 2014. Engineered zinc-finger transcription factors activate OCT4 (POU5F1), SOX2, KLF4, c-MYC (MYC) and miR302/367. Nucleic Acids Research, 42 (10): 6158-6167 (2014) [32].

### A. Tetrazine Chemistry

Tetrazine follows a bioorthogonal ligation chemistry with rapid kinetics that does not depend on a metal catalyst and has been applied to nucleic acid related reactions. Blackman et al., “Tetrazine Ligation: Fast Bioconjugation Based on Inverse-Electron-Demand Diels-Alder Reactivity” J. Am. Chem. Soc. 130 (41): 13518-13519 (2008) [33]; Devaraj et al., “Tetrazine-derived Cycloadditions: Application to Pretargeted Live Cell Imaging” Bioconjugate Chem. 19 (12): 2297-2299 (2008) [34]; Devaraj et al., “Fast and Sensitive Pretargeted Labeling of Cancer Cells through a Tetrazine/Trans-Cyclooctene Cycloaddition” Angewandte Chemie International Edition 48 (38): 7013-7016 (2009) [35]; Knall et al., “Inverse Electron Demand Diels-Alder (IEDDA)-Initiated Conjugation: A (High) Potential Click Chemistry Scheme” Chem. Soc. Rev. 42 (12): 5131 (2013) [36]; Oliveira et al., “Inverse Electron Demand Diels-Alder Reactions in Chemical Biology” Chem. Soc. Rev. 46 (16): 4895-4950 (2017) [37]; Schoch et al., “Post-Synthetic Modification of DNA by Inverse-Electron-Demand Diels-Alder Reaction” J. Am. Chem. Soc. 132 (26): 8846-8847 (2010) [38]; Schoch et al., “Inverse Electron-Demand Diels-Alder Reactions for the Selective and Efficient Labeling of RNA” Chem. Commun. 47 (46): 12536-12537 (2011) [39]; Schoch et al., “Site-Specific One-Pot Dual Labeling of DNA by Orthogonal Cycloaddition Chemistry” Bioconjugate Chem. 23 (7): 1382-1386 (2012) [40]; Šečkutė et al., “Rapid Oligonucleotide-Templated Fluorogenic Tetrazine Ligations” Nucleic Acids Research 41 (15): e148-e148 (2013) [41]; Asare-Okai et al., “Site-Specific Fluorescence Labelling of RNA Using Bio-Orthogonal Reaction of Trans-Cyclooctene and Tetrazine” Chem. Commun. 50 (58): 7844-7847 (2014) [42]; Pyka et al., “Diels-Alder Cycloadditions on Synthetic RNA in Mammalian Cells” Bioconjugate Chem. 25 (8): 1438-1443 (2014) [43]; Domnick et al., “Site-Specific Enzymatic Introduction of a Norbornene Modified Unnatural Base into RNA and Application in Post-Transcriptional Labeling” Chem. Commun. 51 (39): 8253-8256 (2015) [44]; Holstein et al., “Current Covalent Modification Methods for Detecting RNA in Fixed and Living Cells” Methods 98:18-25 (2016) [45]; and Winz et al., “Site-Specific One-Pot Triple Click Labeling for DNA and RNA” Chem. Commun. 54 (83): 11781-11784 (2018) [46].

Tetrazine participates in an inverse electron demand Diels-Alder (IEDDA) reaction with a dienophile; a subsequent retro-Diels-Alder reaction leads to loss of a molecule of N₂ and renders the ligation reaction irreversible.

### B. Tetrazine-Derived Linkers

In one embodiment, the present invention contemplates an sgRNA comprising a tetrazine-derived linker molecule. In one embodiment, the tetrazine-derived linker is conjugated between a crRNA and a tracrRNA. Although it is not necessary to understand the mechanism of an invention, it is believed that tetrazine-ligated sgRNAs provide more efficient and simplified synthesis as opposed to conventional sgRNAs with tetraloop linkers. Despite the general knowledge of tetrazine-derived reactions applied to both DNA and RNA as substrates, tetrazine-derived chemistry has not been used to produce sgRNA molecules by linking a crRNA molecule to a tracrRNA molecule.

### C. Tetrazine-Derived Linker Synthesis

Tetrazine-based inverse electron demand Diels-Alder (IEDDA) reaction has emerged as a promising bioorthogonal ligation chemistry with rapid kinetics that does not depend on a metal catalyst [33-37]. See, FIG. 1A. Despite many examples of nucleic acid applications, the tetrazine-based IEDDA reaction has not been used to produce long RNA molecules [37-46]. As disclosed herein, tetrazine-based ligation of crRNA and tracrRNA can be used to form sgRNAs that support efficient genome editing.

Conventional GAAA linkers form a tetraloop that protrudes from the nuclease in CRISPR-Cas9 structures [22-24]. This suggests that Cas9 nucleases can accommodate structural changes within this tetraloop. In one embodiment, ligation of a tetrazine-derived linker is located at a junction between the 3′-end of crRNA and the 5′-end of tracrRNA. See, FIG. 1B.

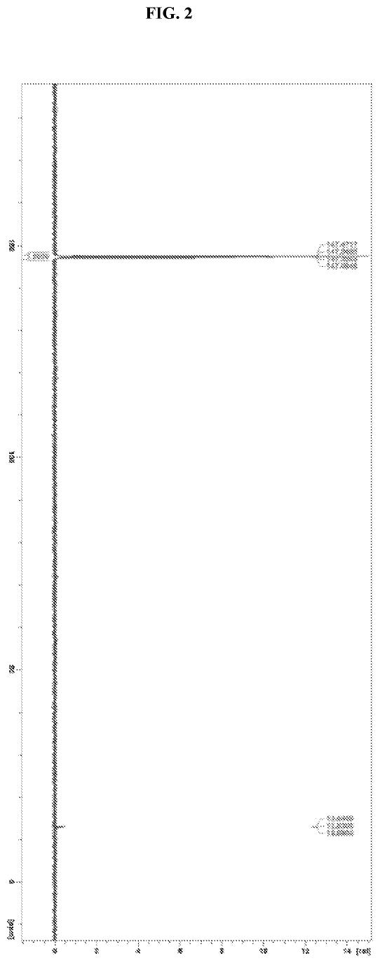

The data presented herein utilizes norbornene as a representative alkene (e.g., a dienophile) that ligates with a tetrazine to create the presently disclosed sgRNA comprising a tetrazine-derived linker molecule. Norbornene has been reported to be readily incorporated into solid-phase RNA synthesis. Schoch et al., “Post-Synthetic Modification of DNA by Inverse-Electron-Demand Diels-Alder Reaction” J. Am. Chem. Soc. 132 (26): 8846-8847 (2010) [38]. Norbornene phosphoramidites were successfully synthesized and conjugated at the 5′-end of tracrRNA during solid-phase synthesis. See, FIG. 2. Other suitable dienophiles include bicyclo[6.1.0] nonyne, trans-cyclooctene, and still others that will be known to a person skilled in the art.

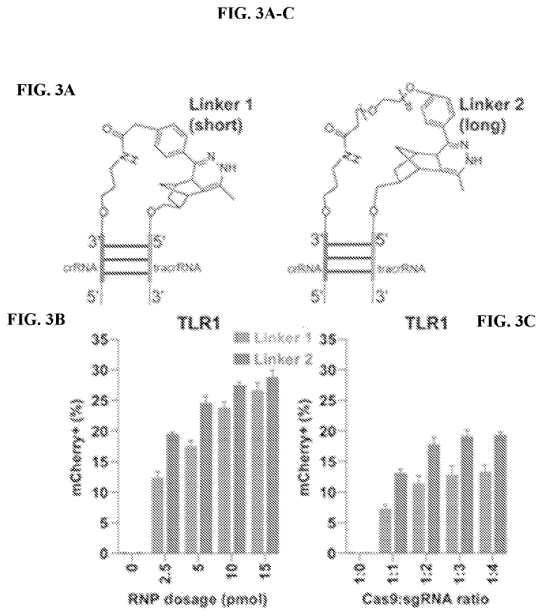

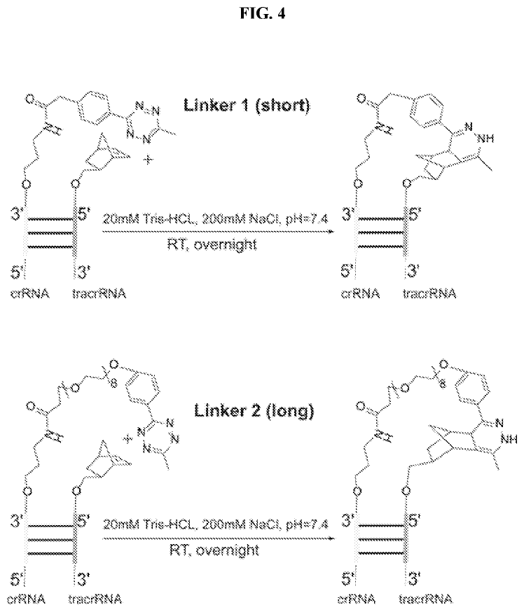

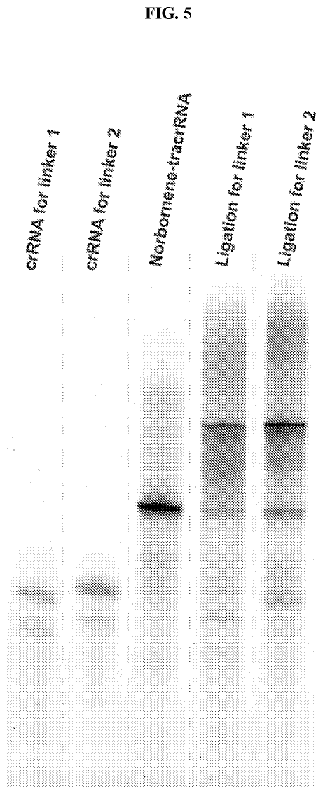

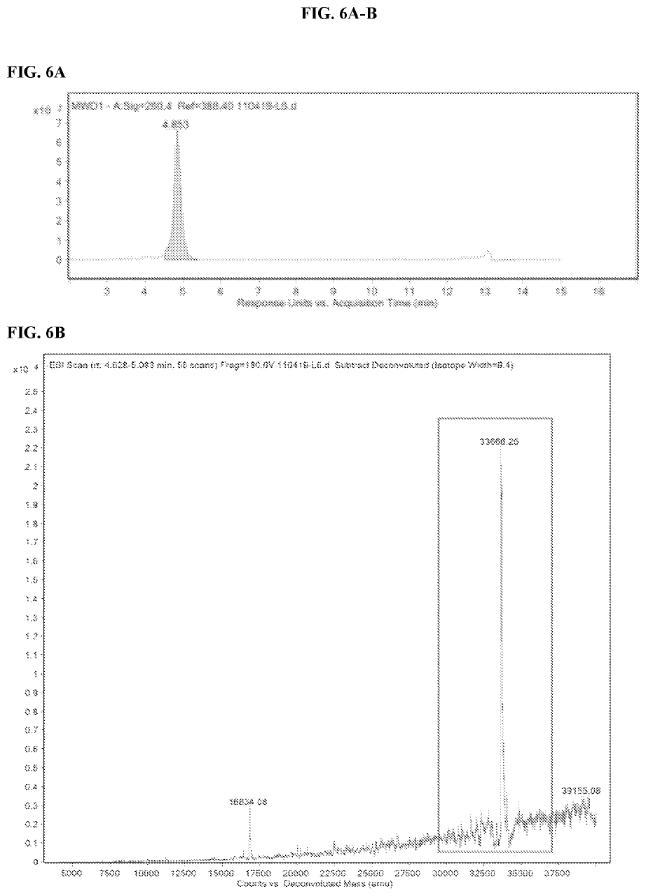

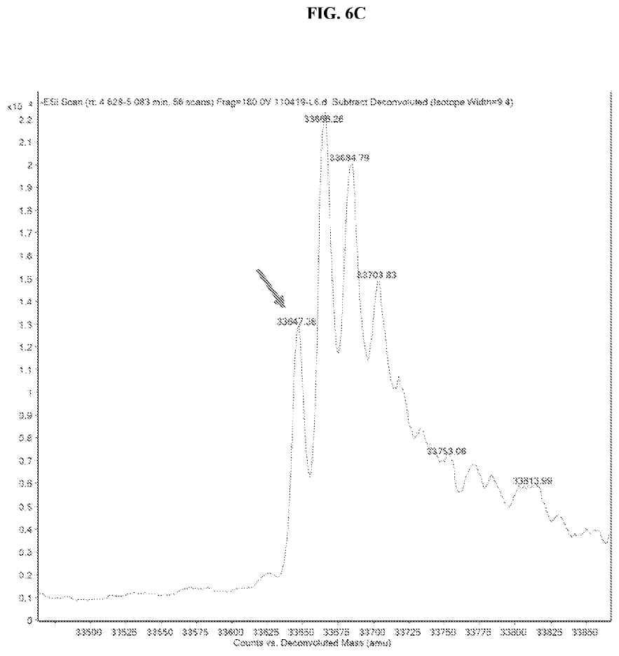

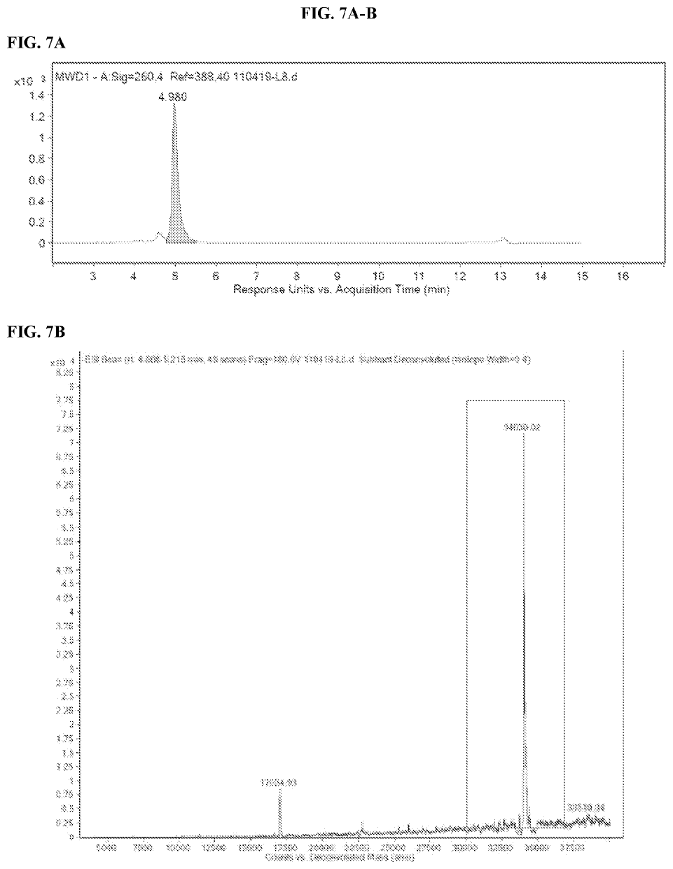

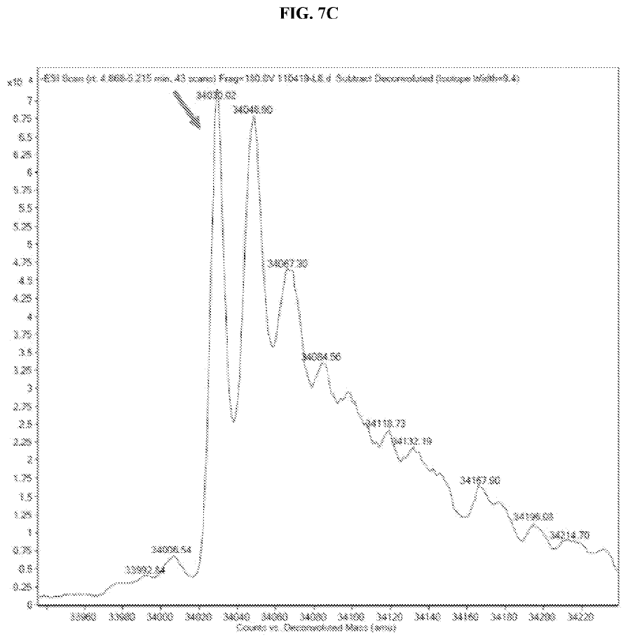

To generate a tetrazine moiety on the crRNA, a 3′-amino modified crRNA was synthesized and conjugated to a tetrazine moiety with a tetrazine NHS ester as substrate. Because the structure of the linker may affect binding of a tetrazine-ligated sgRNA to Cas9, two different tetrazine NHS esters were compared; a short linker 1 or a long linker 2. Long linker 2 includes an extra PEG8 spacer as compared to the short linker 1. See, FIG. 3A. Linker 2 may also include an extra PEG4 spacer. To perform the tetrazine ligation, a 3′-tetrazine-modified crRNA was combined with a 5′-norbornene-modified tracrRNA and incubated under mild conditions (20 mM Tris-HCl, 200 mM NaCl, pH 7.4) for ˜20 hours at room temperature. See, FIG. 4. The success of the ligation reactions were verified by PAGE analysis. FIG. 5. The identities of purified ligation products were then confirmed by HPLC-MS. See, Table 1, and FIGS. 6 & 7.

TABLE 1
Summary of representative tetrazine-ligated sgRNAs.

| | | | Calculated | Observed |
|---|---|---|---|---|
| Locus | Linker | Tetrazine-ligated sgRNA sequences and modifications | MW (Da) | MW (Da) |
| TLR1 | Linker 1 | (mG)(mG)(mU)(rG)(rA)(rG)(rC)(rU)(rC)(rU)(rU)(rA)(rU)(rU)(rU)(rG)(rC)(rG) | 33646.16 | 33647.38 |
| | | (rU)(rA)(rG)(rU)(rU)(rU)(rU)(rA)(rG)(rA)(rG)(rC)(rU)(rA)(rU)(rG)(rC)(rU) | | |
| | | -(Tetrazine)-(Norbornene)- | | |
| | | (rA)(rG)(rC)(rA)(rU)(rA)(rG)(rC)(rA)(rA)(rG)(rU)(rU)(rA)(rA)(rA)(rA)(rU)(rA) | | |
| | | (rA)(rG)(rG)(rC)(rU)(rA)(rG)(rU)(rC)(rC)(rG)(rU)(rU)(rA)(rU)(rC)(rA)(rA)(rC) | | |
| | | (rU)(rU)(rG)(rA)(rA)(rA)(rA)(rA)(rG)(rU)(rG)(rG)(rC)(rA)(rC)(rC)(rG)(rA)(rG) | | |
| | | (rU)(rC)(rG)(rG)(rU)(rG)(rC)(mU)(mU)(mU) | | |
| | | (SEQ ID NO: 1) | | |
| TLR1 | Linker 2 | (mG)(mG)(mU)(rG)(rA)(rG)(rC)(rU)(rC)(rU)(rU)(rA)(rU)(rU)(rU)(rG)(rC)(rG) | 34028.60 | 34030.02 |
| | | (rU)(rA)(rG)(rU)(rU)(rU)(rU)(rA)(rG)(rA)(rG)(rC)(rU)(rA)(rU)(rG)(rC)(rU) | | |
| | | -(PEG8-tetrazine)-(Norbornene)- | | |
| | | (rA)(rG)(rC)(rA)(rU)(rA)(rG)(rC)(rA)(rA)(rG)(rU)(rU)(rA)(rA)(rA)(rA)(rU)(rA) | | |
| | | (rA)(rG)(rG)(rC)(rU)(rA)(rG)(rU)(rC)(rC)(rG)(rU)(rU)(rA)(rU)(rC)(rA)(rA)(rC) | | |
| | | (rU)(rU)(rG)(rA)(rA)(rA)(rA)(rA)(rG)(rU)(rG)(rG)(rC)(rA)(rC)(rC)(rG)(rA)(rG) | | |
| | | (rU)(rC)(rG)(rG)(rU)(rG)(rC)(mU)(mU)(mU) | | |
| | | (SEQ ID NO: 2) | | |
| TLR1 | Linker 2 | (mG)# (mG)#(mU)#(rG)(rA)(rG)(rC)(rU)(rC)(rU)(rU)(rA)(rU)(rU)(rU)(rG)(rC) | 34125.00 | 34127.79 |
| | | (rG)(rU)(rA)(rG)(rU)(rU)(rU)(rU)(rA)(rG)(rA)(rG)(rC)(rU)(rA)(rU)(rG)(rC)(rU) | | |
| | | -(PEG8-tetrazine)-(Norbornene)- | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 3) | | |
| CCR5 | Linker 2 | (mG)# (mC)#(mA)#(rG)(rC)(rA)(rU)(rA)(rG)(rU)(rG)(rA)(rG)(rC)(rC)(rC)(rA) | 34215.19 | 34217.61 |
| | | (rG)(rA)(rA)(rG)(rU)(rU)(rU)(rU)(rA)(rG)(rA)(rG)(rC)(rU)(rA)(rU)(rG)(rC)(rU) | | |
| | | -(PEG8-tetrazine)-(Norbornene)- | | |
| | | (rA)(rG)(rC)(rA)(rU)(rA)(rG)(rC)(rA)(rA)(rG)(rU)(rU)(rA)(rA)(rA)[rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 4) | | |
| HEK3 | Linker 2 | (mG)#(mG)# (mC)# (rC)(rC)(rA)(rG)(rA)(rC)(rT)(rG)(rA)(rG)(rC)(rA)(rC)(rG) | 34207.16 | 34211.29 |
| | | (rU)(rG)(rA)(rG)(rU)(rU)(rU)(rU)(rA)(rG)(rA)(rG)(rC)(rU)(rA)(rU)(rG)(rC)(rU) | | |
| | | -(PEG8-tetrazine)-(Norbornene)- | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 5) | | |
| TRAC | Linker 2 | (mG)# (mA)# (mG)#(rA)(rA)(rU)(rC)(rA)(rA)(rA)(rA)(rU)(rC)(rG)(rG)(rU)(rG) | 34225.19 | 34226.66 |
| | | (rA)(rA)(rU)(rG)(rU)(rU)(rU)(rU)(rA)(rG)(rA)(rG)(rC)(rU)(rA)(rU)(rG)(rC)(rU) | | |
| | | -(PEG8-tetrazine)-(Norbornene)- | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 6) | | |
| HPRT | Linker 2 | (mG)# (mG)#(mU)#(rC)(rA)(rC)(rU)(rU)(rU)(rU)(rA)(rA)(rC)(rA)(rC)(rA)(rC) | 34034.02 | 34037.27 |
| | | (rC)(rC)(rA)(rG)(rU)(rU)(rU)(rU)(rA)(rG)(rA)(rG)(rC)(rU)(rA)(rU)(rG)(rC)(rU) | | |
| | | -(PEG8-tetrazine)-(Norbornene)- | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 7) | | |
| HPRT | Linker 3 | (mG)#(mG)#(mU)#(rC)(rA)(rC)(rU)(rU)(rU)(rU)(rA)(rA)(rC)(rA)(rC)(rA)(rC) | 34163.96 | 34166.15 |
| | | (rC)(rC)(rA)(rG)(rU)(rU)(rU)(rU)(rA)(rG)(rA)(rG)(rC)(rU)(rA)(rU)(rG)(rC)(rU) | | |
| | | -(PEG4)-(Tetrazine)-(Norbornene)-(PEG4)- | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 8) | | |
| HPRT | Linker 4 | (mG)# (mG)# (mU)# (rC)(rA)(rC)(rU)(rU)(rU)(rU)(rA)(rA)(rC)(rA)(rC)(rA)(rC) | 34546.40 | 34548.19 |
| | | (rC)(rC)(rA)(rG)(rU)(rU)(rU)(rU)(rA)(rG)(rA)(rG)(rC)(rU)(rA)(rU)(rG)(rC)(rU) | | |
| | | -(PEG4)-(PEG8-Tetrazine)-(Norbornene)-(PEG4)- | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 9) | | |
| HPRT | Linker 5 | (mG)# (mG)# (mU)#(rC)(rA)(rC)(rU)(rU)(rU)(rU)(rA)(rA)(rC)(rA)(rC)(rA)(rC) | 35587.74 | 35588.85 |
| | | (rc)(rC)(rA)(rG)(rU)(rU)(rU)(rU)(rA)(rG)(rA)(rG)(rC)(rU)(rA)(rU)(rG)(rC)(rU) | | |
| | | (rA)(rC)(rG)-(Tetrazine)-(Norbornene)-(rC)(rG)(rU) | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 10) | | |
| HPRT | Linker 6 | (mG)#(mG)#(mU)# (rC)(rA)(rC)(rU)(rU)(rU)(rU)(rA)(rA)(rC)(rA)(rC)(rA)(rC) | 35970.18 | 35972.12 |
| | | (rC)(rC)(rA)(rG)(rU)(rU)(rU)(rU)(rA)(rG)(rA)(rG)(rC)(rU)(rA)(rU)(rG)(rC)(rU) | | |
| | | (rA)(rC)(rG)-(PEG8-Tetrazine)-(Norbornene)-(rC)(rG)(rU) | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 11) | | |
| TLR1 | Linker 5 | (mG)#(mG)# (mU)#(rG)(rA)(rG)(rC)(rU)(rC)(rU)(rU)(rA)(rU)(rU)(rU)(rG)(rC) | 35678.72 | 35681.19 |
| | | (rG)(rU)(rA)(rG)(rU)(rU)(rU)(rU)(rA)(rG)(rA)(rG)(rC)(rU)(rA)(rU)(rG)(rC)(rU) | | |
| | | (rA)(rC)(rG)-(Tetrazine)-(Norbornene)-(rC)(rG)(rU) | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 12) | | |
| CCR5 | Linker 5 | (mG)# (mC)# (mA)#(rG)(rC)(rA)(rU)(rA)(rG)(rU)(rG)(rA)(rG)(rC)(rC)(rC)(rA) | 35768.91 | 35770.41 |
| | | (rG)(rA)(rA)(rG)(rU)(rU)(rU)(rU)(rA)(rG)(rA)(rG)(rC)(rU)(rA)(rU)(rG)(rC)(rU) | | |
| | | (rA)(rC)(rG)-(Tetrazine)-(Norbornene)-(rC)(rG)(rU) | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 13) | | |
| TRAC | Linker 5 | (mG)#(mA)#(mG)#(rA)(rA)(rU)(rC)(rA)(rA)(rA)(rA)(rU)(rC)(rG)(rG)(rU)(rG) | 35778.91 | 35820.95 |
| | | (rA)(rA)(rU)(rG)(rU)(rU)(rU)(rU)(rA)(rG)(rA)(rG)(rC)(rU)(rA)(rU)(rG)(rC)(rU) | | |
| | | (rA)(rC)(rG)-(Tetrazine)-(Norbornene)-(rC)(rG)(rU) | | |
| | | (rA)(rG)(rC)(rA)(rU)(rA)(rG)(rC)(rA)(rA)(rG)(rU)(rU)(rA)(rA)(rA)(rA)(rU) | | |
| | | (rA)(rA)(rG)(rG)(rC)(rU)(rA)(rG)(rU)(rC)(rC)(rG)(rU)(rU)(rA)(rU)(rC)(rA) | | |
| | | (rA)(rC)(rU)(rU)(rG)(rA)(rA)(rA)(rA)(rA)(rG)(rU)(rG)(rG)(rC)(rA)(rC)(rC) | | |
| | | (rG)(rA)(rG)(rU)(rC)(rG)(rG)(rU)(rG)(rC)#(mU)#(mU)#(mU) | | |
| | | (SEQ ID NO: 14) | | |

Linkers are highlighted in red.
R indicates a ribonucleotide (2'-OH).
M indicates a 2′-O-methylated ribonucleotide (2′-Ome).
# indicates a PS linkage.
Underline indicates the 3 extra base pairs on the stem region of linkers 5 and 6.

### D. Tetrazine-Derived Linker Validation

Cells were tested using an sgRNA comprising a tetrazine-derived linker molecule targeted to a traffic light reporter construct, TLR1. Certo et al., “Tracking Genome Engineering Outcome at Individual DNA Breakpoints” Nat Methods 8 (8): 671-676 (2011) [47]. The TLR reporter has a validated guide RNA sequence with good editing efficiency and allows the usage of flow cytometry for easy quantification. Its nature as a gain-of-function assay facilitates low background when scoring gene editing. In this assay, CRISPR-mediated cleavage of TLR1 is repaired by mutagenic end-joining pathways, shifting an out-of-frame mCherry coding region into the correct reading frame in a subset of edited cells, resulting in mCherry expression. The percentage of mCherry-positive cells measured by flow cytometry therefore provides a measure of gene editing efficiency.

### 1. Short Linker 1 Versus Long Linker 2

The editing efficiency of sgRNAs comprising tetrazine-derived linker molecules was assessed by assembling TLR1 sgRNA-SpyCas9 ribonucleoprotein (RNP) which were electroporated into HEK-293T TLR1 reporter cells. TLR 1 activity was compared using sgRNAs with either a short tetrazine-derived linker 1 or a long tetrazine-derived linker 2 at various RNP dosages with a fixed Cas9: sgRNA ratio of 1:3. See, FIGS. 1B & 3B. Various Cas9: sgRNA ratios were also analyzed with a fixed 2.5 pmol RNP dosage. See, FIG. 3C. The sgRNAs with the tetrazine-derived long linker 2 performed better than the sgRNAs with the tetrazine-derived short linker 1 under all conditions tested, especially at low RNP dosages. One possible explanation for this is that at the higher RNP dosages the assay may be near saturation. These results indicate that the sgRNAs with a tetrazine-derived linker molecule support CRISPR-mediated editing and confirm the relevance of tetrazine-derived linker structure to sgRNA activity.

An sgRNA comprising a tetrazine-derived long linker complex 2 was targeted to a human cell endogenous loci and compared to a standard synthetic sgRNA with a GAAA-tetraloop linking crRNA and tracrRNA (an sgRNA with a tetraloop linker). At each end of the sgRNAs, in both the sgRNA with the tetraloop linker and the sgRNA comprising a tetrazine-derived long linker 2, the last three nucleotides were chemically modified with PS linkages and 2′-O-methyl (2′-Ome) groups to protect them from degradation. Hendel et al., “Chemically Modified Guide RNAs Enhance CRISPR-Cas Genome Editing in Human Primary Cells” Nature Biotechnology 33 (9): 985-989 (2015) [18].

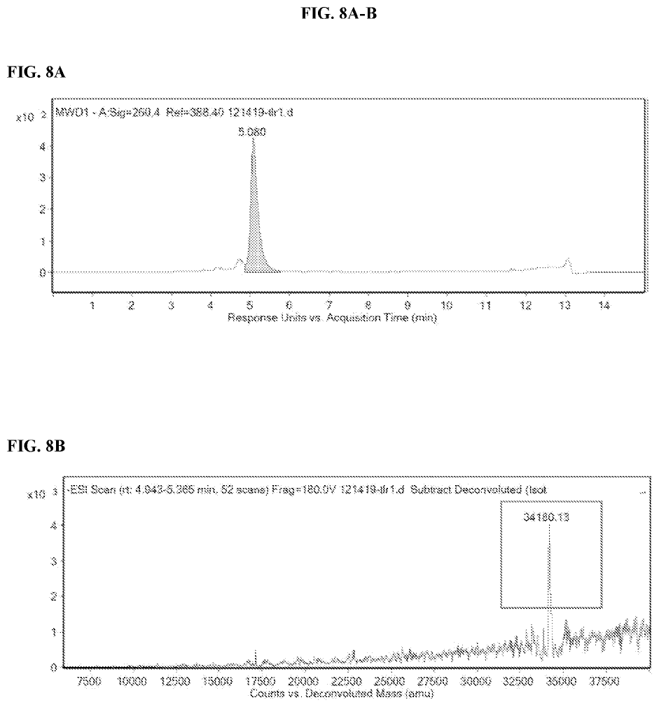

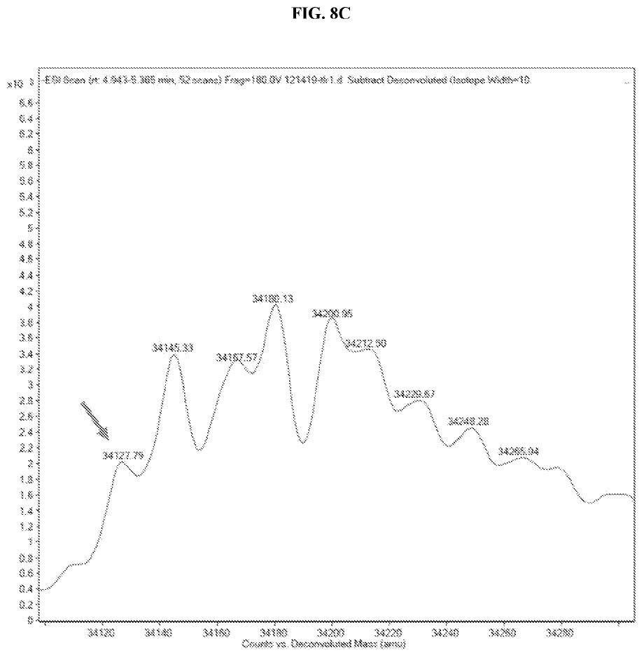

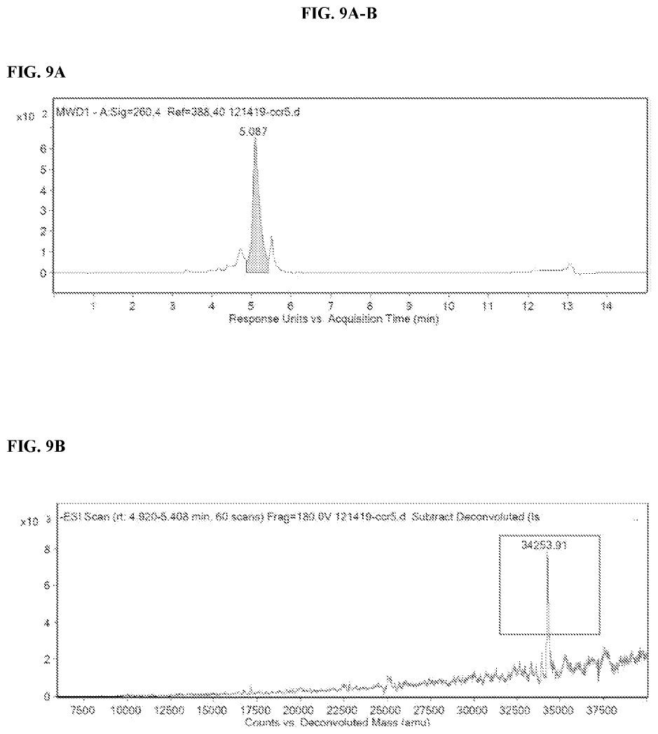

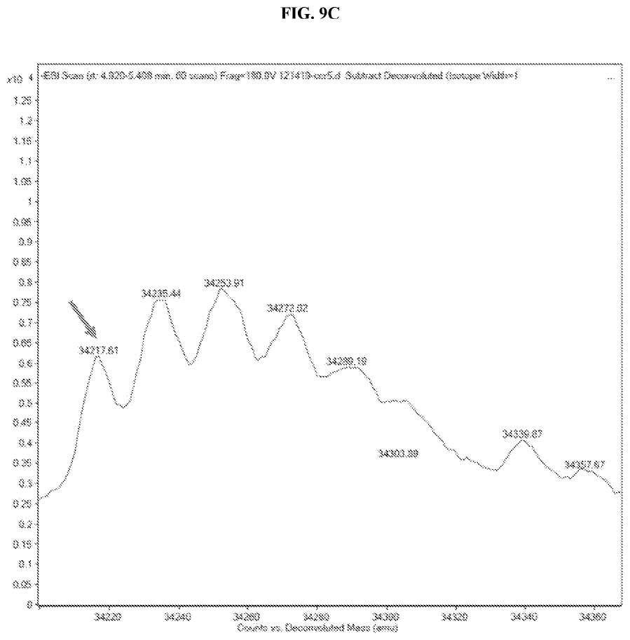

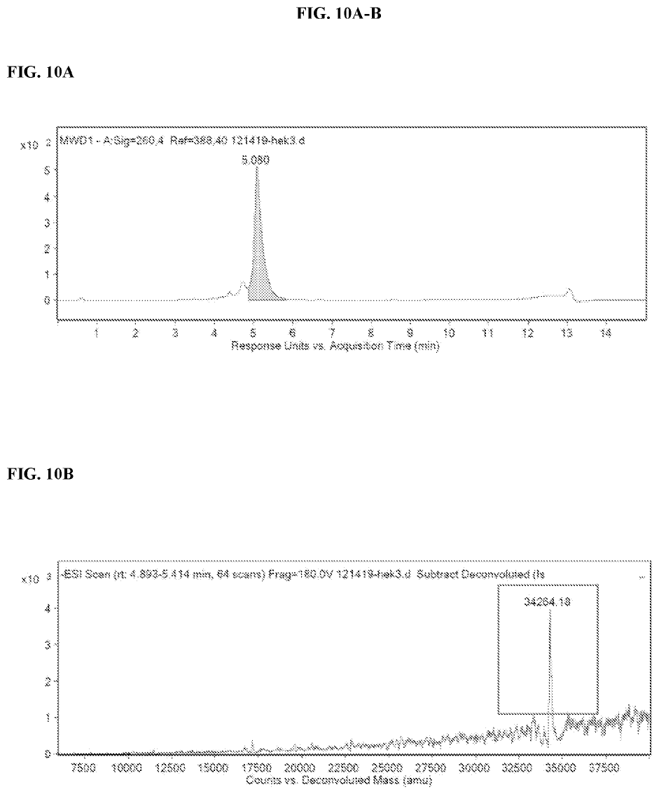

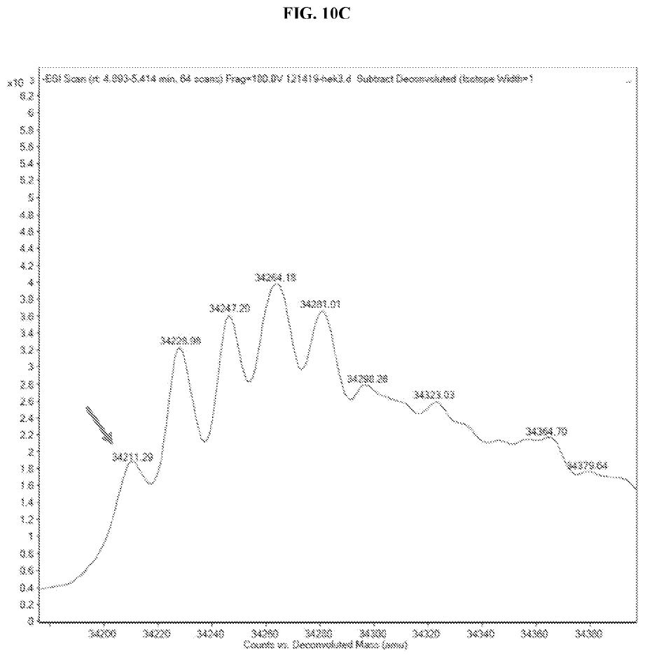

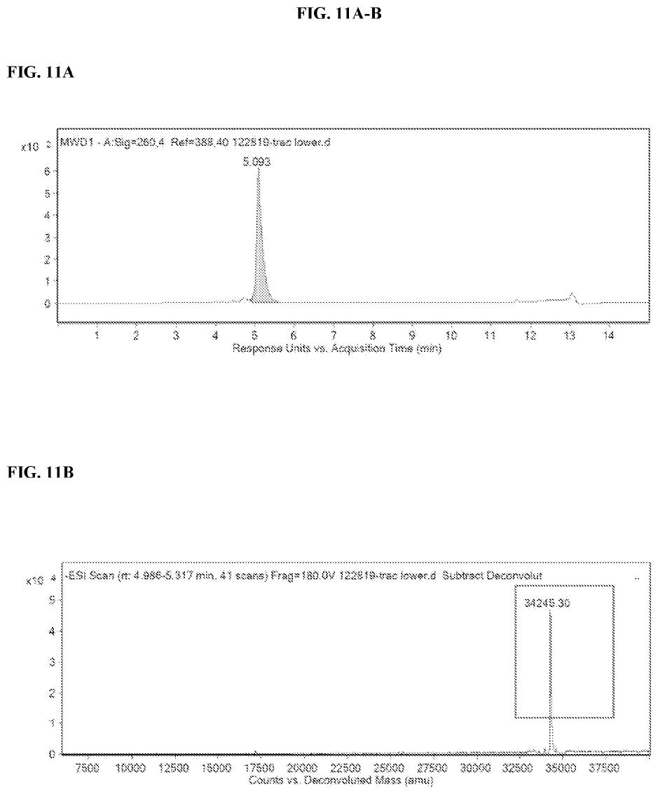

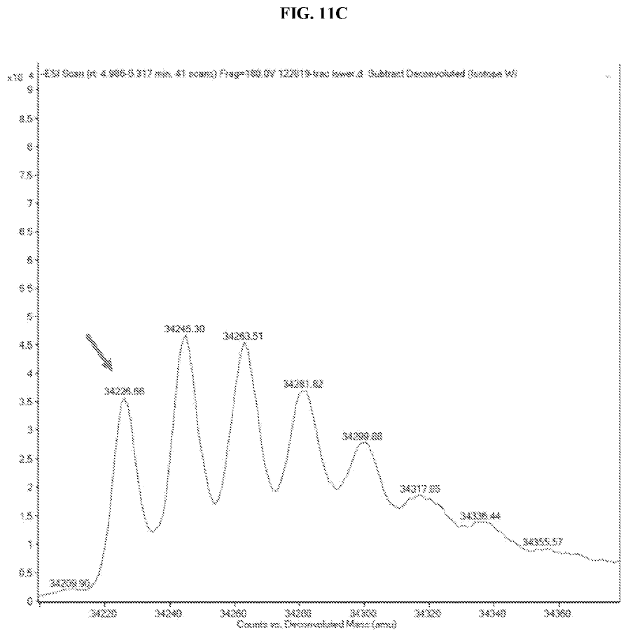

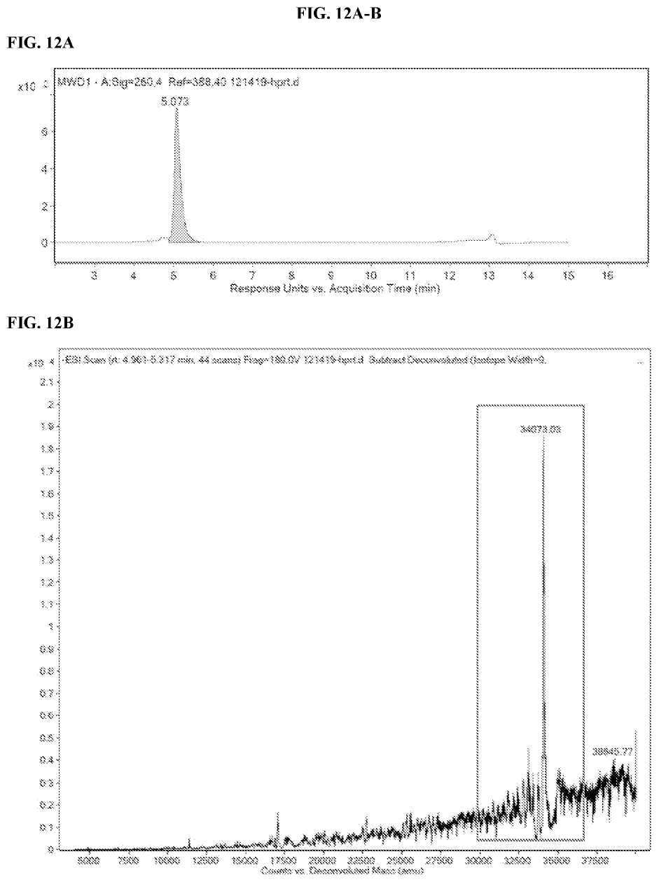

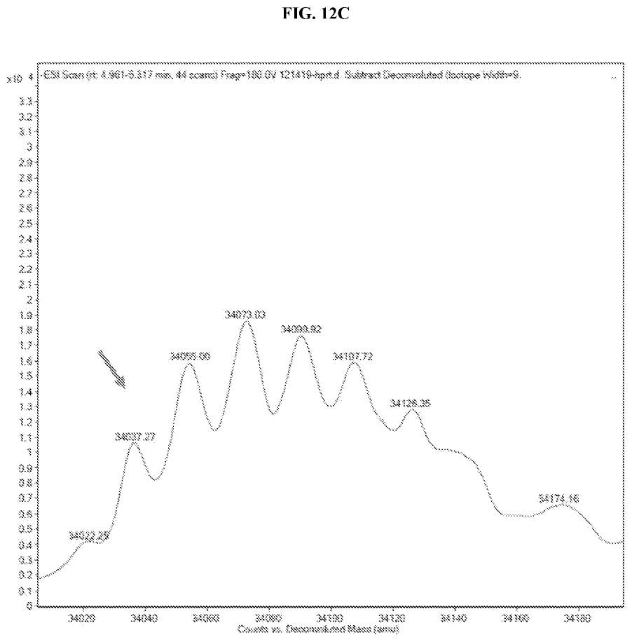

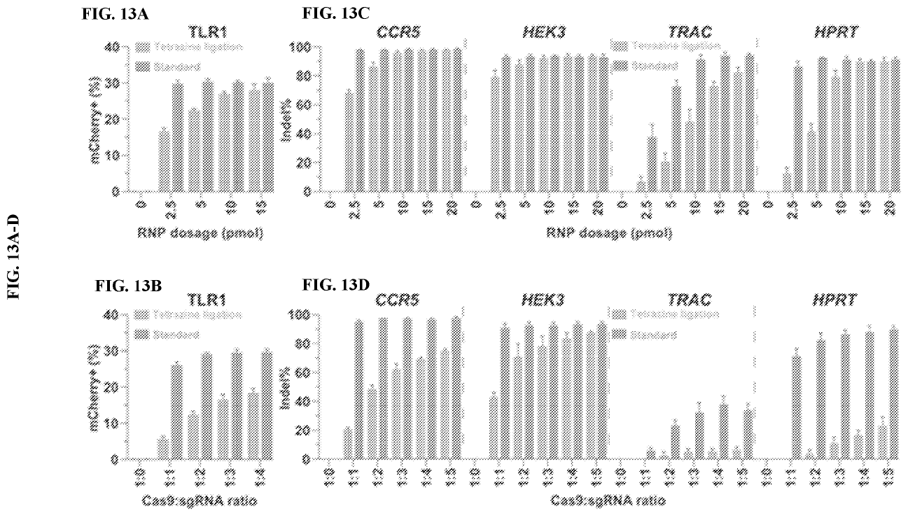

sgRNAs comprising a tetrazine-derived long linker 2 targeting TLR1 and four endogenous loci (CCR5, HEK3, TRAC, and HPRT) were generated, and their identities were confirmed by HPLC-MS. See, Table 1, and FIGS. 8-12. The ability to generate these sgRNAs with a tetrazine-derived long linker 2 indicates that the tetrazine ligation chemistry is generally compatible with chemical modifications (PS, 2′-Ome) that provide sgRNA stability and activity in cells. TLR1 RNPs were electroporated into HEK-293T TLR1 reporter cells to compare sgRNAs with a tetrazine-derived long linker 2 and sgRNAs with a tetraloop linker at: i) various RNP dosages with a fixed 1:3 Cas9: sgRNA ratio; or ii) a fixed 2.5 pmol RNP dosage with various Cas9: sgRNA ratios. See, FIGS. 13A and 13B, respectively. The activity of TLR1 with an sgRNA with a tetrazine-derived long linker 2 was comparable to that of an sgRNA with a tetraloop linker at 10 pmol (27% vs. 30.3%) and 15 pmol (28.1% vs. 30.1) RNP dosage. However, the sgRNA with a tetrazine-derived long linker 2 was less active than the sgRNA with a tetraloop linker at lower RNP dosages (e.g., 16.8% vs 29.8% editing at 2.5 μmol). See, FIG. 13A. The reduced editing activity of TLR1 with an sgRNA with a tetrazine-derived long linker 2 was exacerbated at lower sgRNA: Cas9 ratios. See, FIG. 13B.

To compare the gene editing activities of sgRNAs with a tetrazine-derived linker complex 2 and sgRNAs with a tetraloop linker targeted to endogenous loci, the targeted editing regions were sequenced and gene editing efficiencies were calculated using the ICE algorithm. Hsiau et al., “Inference of CRISPR Edits from Sanger Trace Data” (2022) The CRISPR Journal 5 (1), 123-130 [48]. Consistent with the TLR assay, sgRNA with a tetrazine-derived linker complex 2 and sgRNA with a tetraloop linker targeted to endogenous loci were similarly active at high RNP dosages, but at low RNP dosages the sgRNA with a tetrazine-derived linker complex 2 were less active than an sgRNA with a tetraloop linker. See, FIG. 13C. The editing efficiency of the sgRNA with a tetrazine-derived linker complex 2 was further reduced when the Cas9: sgRNA ratio decreased. See, FIG. 13D. Overall, the sgRNA with a tetrazine-derived long linker 2 consistently performed well at high RNP dosages, but weakly at low RNP dosages.

### 2. Long Linkers 3-6

The above results suggested that sgRNAs with a tetrazine-derived long linker 2 could become more efficient with empirical structural modifications. Consequently, several hypotheses were considered: (i) linker 2 required structural modification to improve flexibility for conformation into RNP; (ii) the PEG8 spacer in linker 2 required spatial reconfiguration; or (iii) detrimental interactions between linker 2 and the Cas9 protein need to be reduced.

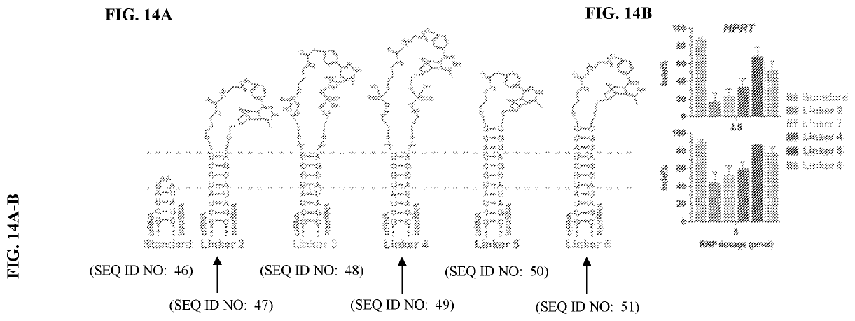

To address these possibilities, alternative tetrazine-derived linkers were designed and synthesized. See, FIG. 14A. For example, tetrazine-derived long linkers 3-6 had the following structural improvements:
i) linker 3 has the same length as linker 2, but instead of a PEG8, a PEG4 spacer was incorporated on each side of the linker.
ii) linker 4 includes the same PEG8 spacer as linker 2 in addition to both PEG4 spacers as in linker 3, adding length and flexibility relative to linker 2.
iii) linker 5 is an analog of linker 1, but the stem formed by the crRNA and tracrRNA is extended by 3 base pairs, based on the hypothesis that the extended (rigid) duplex structure might minimize interactions between the dihydro-pyridazine linkage and Cas9; and
iv) linker 6 is an analog of linker 2, but the stem formed by the crRNA and tracrRNA is extended by 3 base pairs, based on the hypothesis that the extended (rigid) duplex structure might minimize interactions between the dihydro-pyridazine linkage and Cas9.

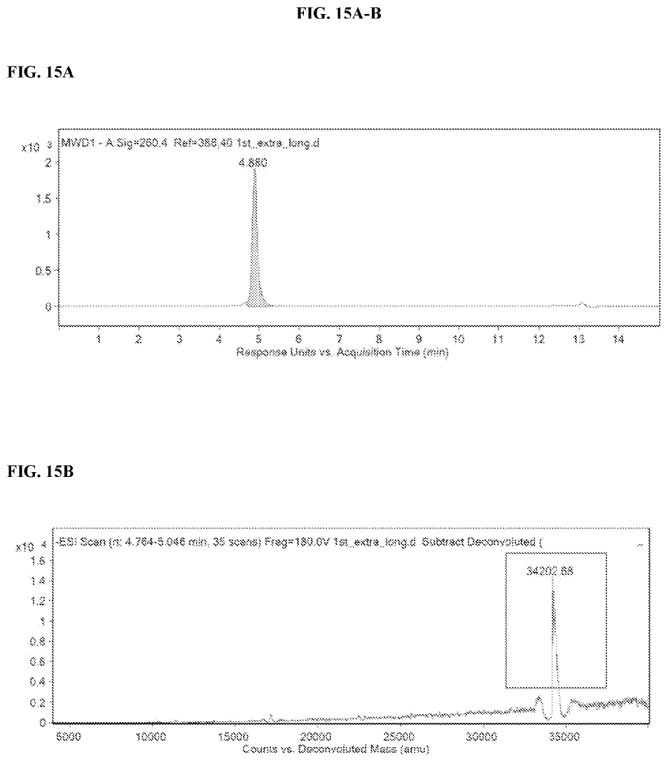

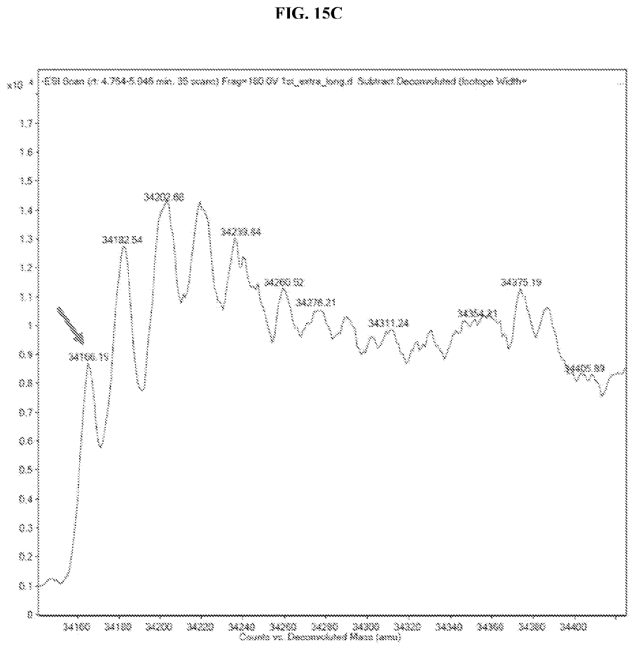

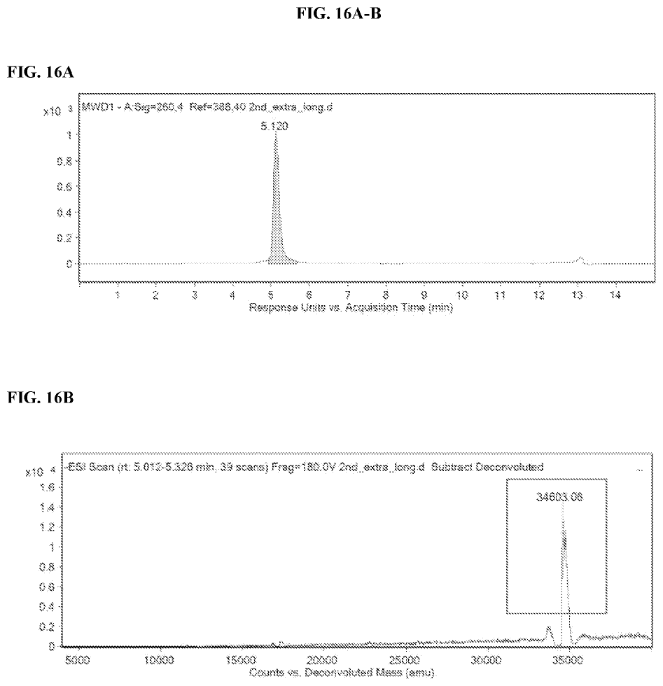

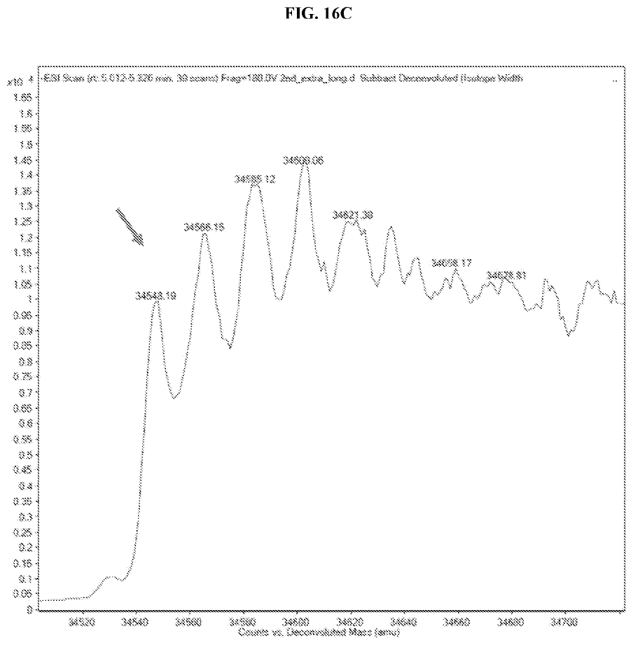

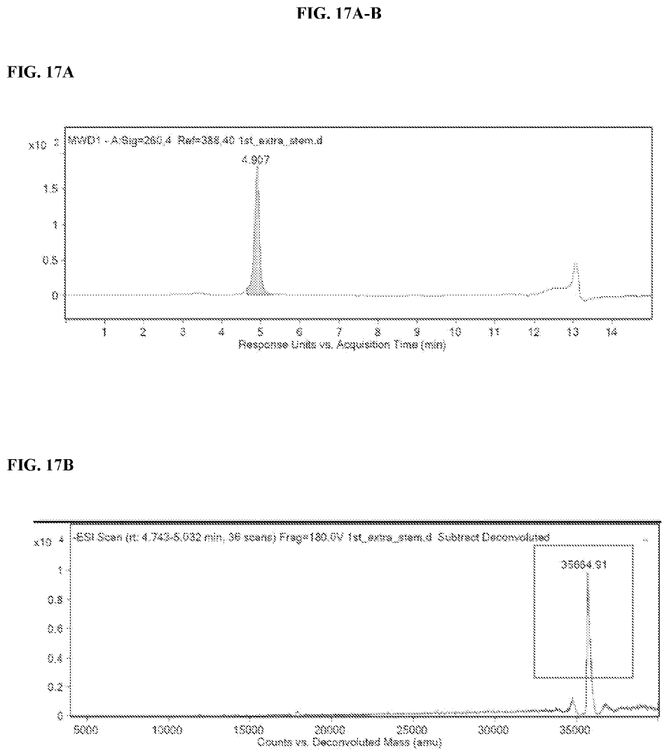

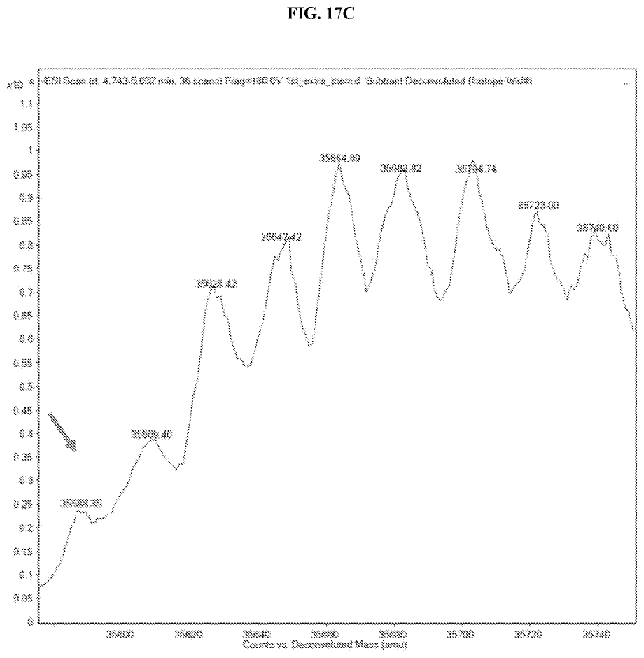

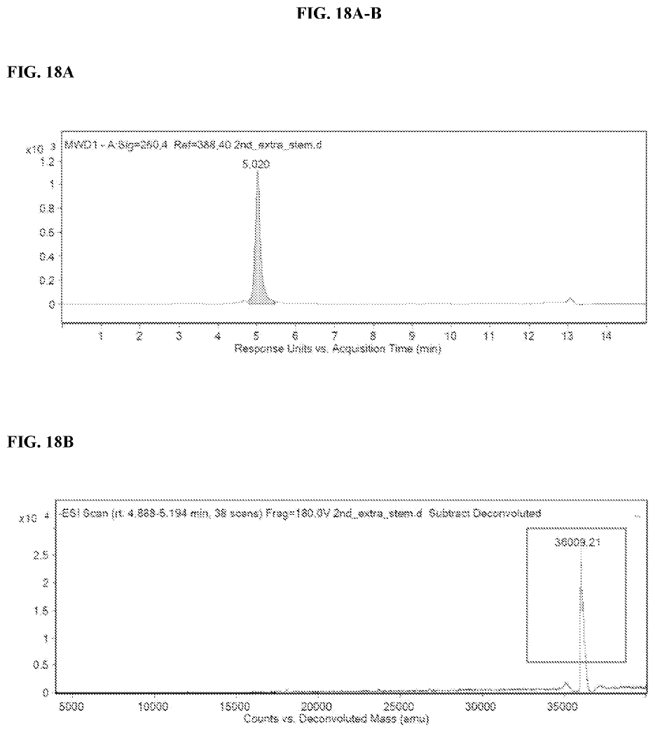

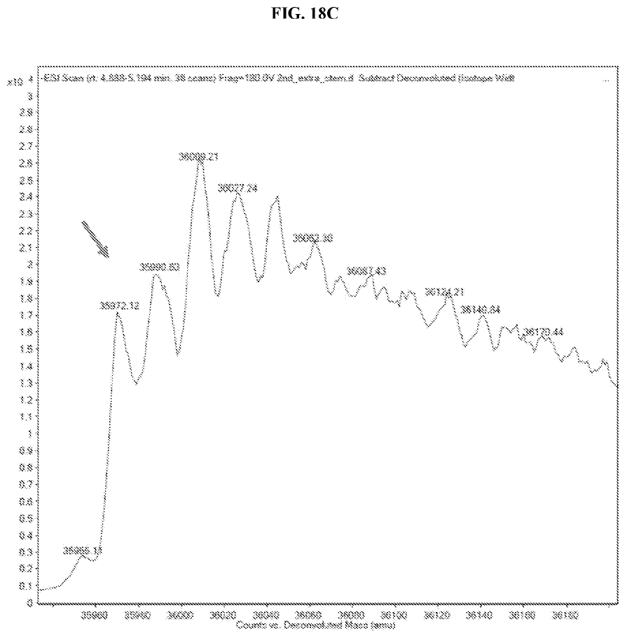

sgRNAs comprising tetrazine-derived long linkers 3-6 were targeted to HPRT, for which the gene editing efficiency difference was greatest between the sgRNA with a tetrazine-derived long linker 2 and the sgRNA with a tetraloop linker. See, FIGS. 13C and 13D. The structures of the sgRNAs with tetrazine-derived long linkers 3 to 6 were confirmed by HPLC-MS and assembled into RNPs. See, Table 1, and FIGS. 15-18.

Gene editing efficiencies were compared between sgRNAs with tetrazine-derived long linkers 2-6 and sgRNAs with a tetraloop linker at 2.5 pmol or 5 pmol RNPs dosages that showed the largest differences in activity between the linker 2 and the tetraloop linker (supra). sgRNAs with tetrazine-derived long linkers 3-6 all performed better at both RNP dosages than did the sgRNAs with a tetrazine-derived long linker 2. See, FIG. 14B. For example, sgRNAs with a tetrazine-derived long linker 5 were ˜ 4 fold more effective (at 2.5 pmol RNP dosage: 67.8% v. 17%) and was only 22% less active than sgRNAs with a tetraloop linker (87.2%). At the 5 pmol RNP dosage, the activity of sgRNAs with a tetrazine-derived long linker 5 (86.7%) was comparable to that of sgRNAs with tetraloop linkers (89.5%). Notably, sgRNAs with a tetrazine-derived long linker 6 was the second most effective. The improved performance of linkers 5 and 6 suggests that extending the crRNA: tracrRNA stem structure improves the activity of sgRNAs with tetrazine-derived linkers.

The above data suggested the possibility that the increment of gene editing efficiency from linker 2 to linker 5 might simply be caused by the extension of the stem itself, unrelated to the linker structure. Prior literature has shown that stem extension can improve the activity of RNP complexes, possibly due to increases in gRNA stability and gRNA-Cas9 assembly [3, 9, 49-52].

Because linker 2 sgRNA has four more base pairs than a standard tetraloop-linked sgRNA in the upper stem region, a GAAA-linked sgRNA control and a dual-guide (dgRNA) control were designed having the same four additional base pairs. Similarly, for linker 5 sgRNA, the GAAA-linked sgRNA and dgRNA controls contained seven more base pairs in the upper stem region. These seven guide designs were then compared across the four loci: TLR1, HPRT, CCR5 and TRAC.

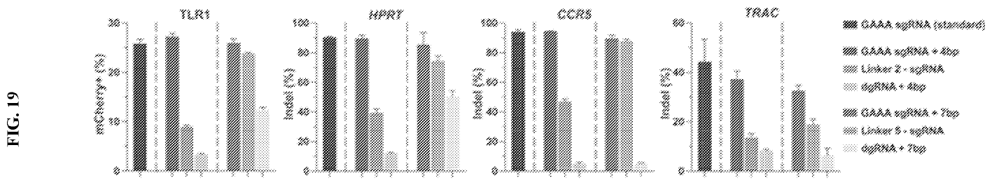

The data showed that Linker 5 sgRNAs provided higher gene editing efficiency than linker 2 sgRNAs at all loci, though the magnitude of improvement varied. See, FIG. 19. For instance, linker 5 sgRNAs were equivalent to GAAA-linked sgRNAs at TLR1, HPRT and CCR5 loci. Moreover, both linker 2 and linker 5 sgRNAs were more active than their corresponding dgRNA controls at all loci. This confirms the importance of sgRNAs for efficient genome editing. Lastly, since the editing efficiency of GAAA sgRNA controls was similar between different stem lengths, this suggests that the improvement from linker 2 to linker 5 is not simply due to the longer stem of linker 5.

The data presented herein suggest that the activity of sgRNAs with tetrazine-derived linker molecules depend on the precise structure of the linker moiety and the context of the crRNA: tracrRNA stem structure. For example, with a shorter stem, long linker 2 sgRNA was more active than short linker 1 sgRNA. See, FIGS. 3B-3C. Conversely, when the crRNA: tracrRNA stem was extended by 3 base pairs, the linker 5 sgRNA was more active than linker 6 sgRNA. See, FIG. 14B. Although it is not necessary to understand the mechanism of an invention, it is believed that, in the context of a shorter stem, conformational constraints of the less flexible (i.e., shorter) linker may result in reduced gene editing efficiencies. In contrast, a longer stem structure may be more likely to maintain an appropriate conformation, while a shorter and less flexible linker may have an advantage in reducing interactions with Cas9.

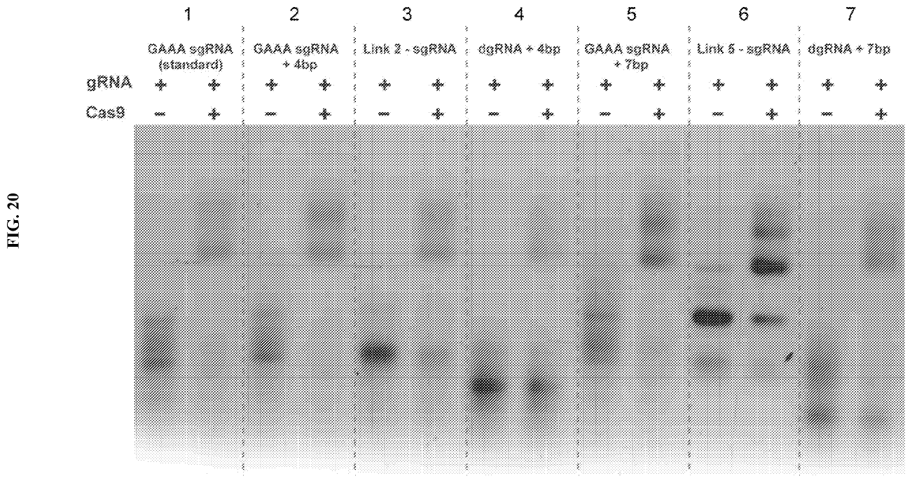

Although it is not necessary to understand the mechanism of an invention, it is believed that pyridazine-based linkages, particularly in the context of short stem lengths, may impair optimal interactions with Cas9. As shown in an electrophoretic mobility shift assay (EMSA) for the above seven TLR1 gRNAs the results suggested that sgRNAs with a tetrazine-derived linkage had somewhat lower affinities for Cas9 than the corresponding GAAA-linked sgRNAs. See, FIG. 20. Note that the sgRNAs with either tetrazine-derived linker 2 or linker 5 (sets 3 and 6 above, respectively) and the dual guide sgRNAs (sets 4 and 7 above) show more unbound guide RNA in the presence of equivalent Cas9 relative to the standard or “extended standard” sgRNAs (sets 1, 2 and 5 above). Incidentally, this gel also confirms that the slightly lower activity is not due to lower purity-if anything, the sgRNAs with tetrazine-derived linker molecules (sets 3 and 6) are of higher purity than the sgRNAs with tetraloop linkers obtained from a commercial source, which reflects the current challenge of synthesizing very long RNA guides and emphasizes one advantage of the presently disclosed tetrazine-based ligation approach to synthesis.

### EXPERIMENTAL

### Example I

### Materials

All reagents and solvents were obtained from commercial sources and used without any further purification. Reaction progress was monitored by thin layer chromatography TLC. Compounds were purified by automated column chromatography (Biotage) unless otherwise noted. Ethyl acetate: hexane: 0.1% triethylamine was used as the eluent for column chromatography. NMR spectra for all compounds were obtained on a 500 MHz Bruker spectrometer. SpyCas9 proteins were purchased from QB3-MacroLab, UC-Berkeley. Standard GAAA-tetraloop sgRNAs were purchased from Integrated DNA Technologies (IDT).

### Example II

### RNA Oligonucleotide Synthesis

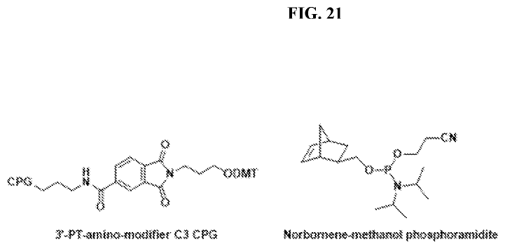

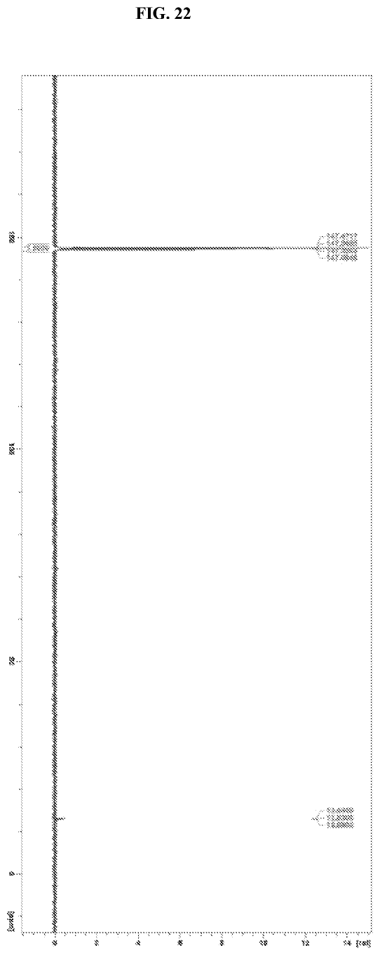

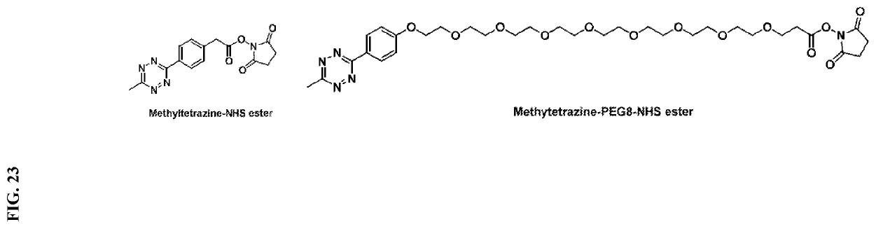

RNAs were synthesized on commercially available ABI 394 or Dr. Oligo 48 DNA synthesizers. To obtain amine functionality at the 3′-end of all crRNAs (both short and long stem versions), synthesis was performed on 3′-phthalimide (PT)-amino-modifier C3 controlled pore glass (CPG), purchased from Glen Research. See, FIG. 21. Norbornene-methanol phosphoramidite was synthesized as described below and then introduced at the 5′-end of tracrRNAs (both short and long stem version) during in-line synthesis. PEG4 was introduced at the 3′-end of crRNA and 5′-end of tracrRNA as needed using commercially available phosphoramidites (ChemGenes). Cleavage and nucleobase/phosphate deprotection were achieved by AMA treatment at 65° C. for 20 min, followed by 2′-O silyl deprotection at 65° C. for 1.5 h with TEA•3HF and DMSO. RNAs were precipitated with t-BuOH, purified by semi-preparative RP-HPLC and were characterized by HPLC-MS. See, Table 2.

TABLE 2
Summary of representative crRNAs and tracrRNAs used for ligation.

| | | RNA sequences and modifications (5'-3') | Calculated | Observed |
|---|---|---|---|---|
| Locus | RNAs | | MW (Da) | MW (Da) |
| TLR1 | crRNA-C3-amino, no PS | (mG)(mG)(mU)(rG)(rA)(rG)(rC)(rU)(rC)(rU)(rU) | 11662.90 | 11662.40 |
| | | (rA)(rU)(rU)(rU)(rG)(rC)(rG)(rU)(rA)(rG)(rU)(rU) | | |
| | | (rU)(rU)(rA)(rG)(rA)(rG)(rC)(rU)(rA)(rU)(rG)(rC) | | |
| | | (rU)-C3-NH2 | | |
| | | (SEQ ID NO: 15) | | |
| | crRNA-tetrazine, no PS | (mG)(mG)(mU)(rG)(rA)(rG)(rC)(rU)(rC)(rU)(rU) | 11875.94 | 11875.96 |
| | | (rA)(rU)(rU)(rU)(rG)(rC)(rG)(rU)(rA)(rG)(rU)(rU) | | |
| | | (rU)(rU)(rA)(rG)(rA)(rG)(rC)(rU)(rA)(rU)(rG)(rC) | | |
| | | (rU)-tetrazine | | |
| | | (SEQ ID NO: 16) | | |
| | crRNA-(PEG8-tetrazine), no PS | (mG)(mG)(mU)(rG)(rA)(rG)(rC)(rU)(rC)(rU)(rU) | 12258.43 | 12258.33 |
| | | (rA)(rU)(rU)(rU)(rG)(rC)(rG)(rU)(rA)(rG)(rU)(rU) | | |
| | | (rU)(rU)(rA)(rG)(rA)(rG)(rC)(rU)(rA)(rU)(rG)(rC) | | |
| | | (rU)-(PEG8-tetrazine) | | |
| | | (SEQ ID NO: 17) | | |
| | crRNA-C3-amino, with PS | (mG)#(mG)# (mU)#(rG)(rA)(rG)(rC)(rU)(rC)(rU) | 11711.17 | 11711.92 |
| | | (rU)(rA)(rU)(rU)(rU)(rG)(rC)(rG)(rU)(rA)(rG)(rU) | | |
| | | (rU)(rU)(rU)(rA)(rG)(rA)(rG)(rC)(rU)(rA)(rU)(rG) | | |
| | | (rC)(rU)-C3-NH2 | | |
| | | (SEQ ID NO: 18) | | |
| | crRNA-(PEG8-tetrazine), with PS | (mG)# (mG)# (mU)#(rG)(rA)(rG)(rC)(rU)(rC)(rU) | 12306.70 | 12306.97 |
| | | (rU)(rA)(rU)(rU)(rU)(rG)(rC)(rG)(rU)(rA)(rG)(rU) | | |
| | | (rU)(rU)(rU)(rA)(rG)(rA)(rG)(rC)(rU)(rA)(rU)(rG) | | |
| | | (rC)(rU)-(PEG8-tetrazine) | | |
| | | (SEQ ID NO: 19) | | |
| CCR5 | crRNA-C3-amino | (mG)# (mC)#(mA)#(rG)(rC)(rA)(rU)(rA)(rG)(rU) | 11801.27 | 11802.16 |
| | | (rG)(rA)(rG)(rC)(rC)(rC)(rA)(rG)(rA)(rA)(rG)(rU) | | |
| | | (rU)(rU)(rU)(rA)(rG)(rA)(rG)(rC)(rU)(rA)(rU)(rG) | | |
| | | (rC)(rU)-C3-NH2 | | |
| | | (SEQ ID NO: 20) | | |
| | crRNA-(PEG8-tetrazine) | (mG)# (mC)#(mA)#(rG)(rC)(rA)(rU)(rA)(rG)(rU) | 12397.05 | 12397.14 |
| | | (rG)(rA)(rG)(rC)(rC)(rC)(rA)(rG)(rA)(rA)(rG)(rU) | | |
| | | (rU)(rU)(rU)(rA)(rG)(rA)(rG)(rC)(rU)(rA)(rU)(rG) | | |
| | | (rC)(rU)-(PEG8-tetrazine) | | |
| | | (SEQ ID NO: 21) | | |
| HEK3 | crRNA-C3-amino | (mG)# (mG)# (mC)#(rC)(rC)(rA)(rG)(rA)(rC)(rU) | 11793.27 | 11794.01 |
| | | (rG)(rA)(rG)(rC)(rA)(rC)(rG)(rU)(rG)(rA)(rG)(rU) | | |
| | | (rU)(rU)(rU)(rA)(rG)(rA)(rG)(rC)(rU)(rA)(rU)(rG) | | |
| | | (rC)(rU)-C3-NH2 | | |
| | | (SEQ ID NO: 22) | | |
| | crRNA-(PEG8-tetrazine) | (mG)# (mG)# (mC)#(rC)(rC)(rA)(rG)(rA)(rC)(rU) | 12388.80 | 12389.03 |
| | | (rG)(rA)(rG)(rC)(rA)(rC)(rG)(rU)(rG)(rA)(rG)(rU) | | |
| | | (rU)(rU)(rU)(rA)(rG)(rA)(rG)(rC)(rU)(rA)(rU)(rG) | | |
| | | (rC)(rU)-(PEG8-tetrazine) | | |
| | | (SEQ ID NO: 23) | | |
| TRAC | crRNA-C3-amino | (mG)# (mA)#(mG)#(rA)(rA)(rU)(rC)(rA)(rA)(rA) | 11811.27 | 11812.17 |
| | | (rA)(rU)(rC)(rG)(rG)(rU)(rG)(rA)(rA)(rU)(rG)(rU) | | |
| | | (rU)(rU)(rU)(rA)(rG)(rA)(rG)(rC)(rU)(rA)(rU)(rG) | | |
| | | (rC)(rU)-C3-NH2 | | |
| | | (SEQ ID NO: 24) | | |
| | crRNA-(PEG8-tetrazine) | (mG)#(mA)#(mG)#(rA)(rA)(rU)(rC)(rA)(rA)(rA) | 12406.80 | 12406.80 |
| | | (A)(rU)(rC)(rG)(rG)(rU)(rG)(rA)(rA)(rU)(rG)(rU) | | |
| | | (rU)(rU)(rU)(rA)(rG)(rA)(rG)(rC)(rU)(rA)(rU)(rG) | | |
| | | (rC)(rU)-(PEG8-tetrazine) | | |
| | | (SEQ ID NO: 25) | | |
| HPRT | crRNA-C3-amino | (mG)# (mG)# (mU)#(rC)(rA)(rC)(rU)(rU)(rU)(rU) | 11620.17 | 11620.94 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(rU)-C3-NH2 | | |
| | | (SEQ ID NO: 26) | | |
| | crRNA-PEG8 tetrazine | (mG)# (mG)# (mU)#(rC)(rA)(rC)(rU)(rU)(rU)(rU) | 12215.70 | 12215.37 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(rU)-(PEG8-tetrazine) | | |
| | | (SEQ ID NO: 27) | | |
| | crRNA-PEG4-C3-amino | (mG)#(mG)# (mU)#(rC)(rA)(rC)(rU)(rU)(rU)(rU) | 11876.20 | 11877.26 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(rU)-(PEG4)-C3-NH2 | | |
| | | (SEQ ID NO: 28) | | |
| | crRNA-PEG4-tetrazine | (mG)#(mG)#(mU)#(rC)(rA)(rC)(rU)(rU)(rU)(rU) | 12089.24 | 12089.40 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(rU)-(PEG4)-tetrazine | | |
| | | (SEQ ID NO: 29) | | |
| | crRNA-PEG4-(PEG8-tetrazine) | (mG)# (mG)# (mU)#(rC)(rA)(rC)(rU)(rU)(rU)(rU) | 12471.73 | 12471.95 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(rU)-(PEG4)-(PEG8-tetrazine) | | |
| | | (SEQ ID NO: 30) | | |
| | crRNA-ACG-C3-amino | (mG)#(mG)# (mU)#(rC)(rA)(rC)(rU)(rU)(rU)(rU) | 12599.70 | 12600.71 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(rU)(rA)(rC)(rG)-C3-NH2 | | |
| | | (SEQ ID NO: 31) | | |
| | crRNA-ACG-tetrazine | (mG)#(mG)#(mU)#(rC)(rA)(rC)(rU)(rU)(rU)(rU) | 12812.74 | 12812.71 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(r)(rA)(rC)(rG)-tetrazine | | |
| | | (SEQ ID NO: 32) | | |
| | crRNA-ACG-PEG8 tetrazine | (mG)#(mG)#(mU)#(rC)(rA)(rC)(rU)(rU)(rU)(rU) | 13195.23 | 13195.17 |
| | | (rA)(rA)(rC)(rA)(rC)(rA)(rC)(rC)(rC)(rA)(rG)(rU) | | |
| | | (rU)(rU)(rU)(rA)(rG)(rA)(rG)(rC)(rU)(rA)(rU)(rG) | | |
| | | (rC)(rU)(rA)(rC)(rG)-(PEG8-tetrazine) | | |
| | | (SEQ ID NO: 33) | | |
| tracrRNA | Norbornene-tracrRNA no PS | Norbornene- | 21799.40 | 21799.60 |
| | | (rA)(rG)(rC)(rA)(rU)(rA)(rG)(rC)(rA)(rA)(rG)(rU) | | |
| | | (rU)(rA)(rA)(rA)(rA)(rU)(rA)(rA)(rG) | | |
| | | (rG)(rC)(rU)(rA)(rG)(rU)(rC)(rC)(rG)(rU)(rU)(rA) | | |
| | | (rU)(rC)(rA)(rA)(rC)(rU)(rU)(rG)(rA)(rA)(rA)(rA) | | |
| | | (rA)(rG)(rU)(rG)(rG)(rC)(rA)(rC)(rC)(rG)(rA)(rG) | | |
| | | (rU)(rC)(rG)(rG)(rU)(rG)(rC)(mU)(mU)(mU) | | |
| | | (SEQ ID NO: 34) | | |
| | Norbornene-tracrRNA with PS | Norbornene- | 21847.34 | 21848.63 |
| | | (rA)(rG)(rC)(rA)(rU)(rA)(rG)(rC)(rA)(rA)(rG)(rU) | | |
| | | (rU)(rA)(rA)(rA)(rA)(rU)(rA)(rA)(rG)(rG)(rC) | | |
| | | (rU)(rA)(rG)(rU)(rC)(rC)(rG)(rU)(rU)(rA)(rU)(rC) | | |
| | | (rA)(rA)(rC)(rU)(rU)(rG)(rA)(rA)(rA)(rA)(rA)(rG) | | |
| | | (rU)(rG)(rG)(rC)(rA)(rC)(rC)(rG)(rA)(rG)(rU)(rC) | | |
| | | (rG)(rG)(rU)(rG)(rC)#(mU)#(mU)#(mU) | | |
| | | (SEQ ID NO: 35) | | |
| | Norbornene-PEG4-tracrRNA with PS | Norbornene-(PEG4)- | 22103.35 | 22104.45 |
| | | (rA)(rG)(rC)(rA)(rU)(rA)(rG)(rC)(rA)(rA)(rG)(rU) | | |
| | | (rU)(rA)(rA)(rA)(rA)(rU)(rA)(rA)(rG )(rG)(rC) | | |
| | | (rU)(rA)(rG)(rU)(rC)(rC)(rG)(rU)(rU)(rA)(rU)(rC) | | |
| | | (rA)(rA)(rC)(rU)(rU)(rG)(rA)(rA)(rA)(rA)(rA)(rG) | | |
| | | (rU)(rG)(rG)(rC)(rA)(rC)(rC)(rG)(rA)(rG)(rU)(rC) | | |
| | | (rG)(rG)(rU)(rG)(rC)#(mU)#(mU)#(mU) | | |
| | | (SEQ ID NO: 36) | | |
| | Norbornene-CGU-tracrRNA with PS | Norbornene-(rC)(rG)(rU) | 22803.80 | 22804.70 |
| | | (rA)(rG)(rC)(rA)(rU)(rA)(rG)(rC)(rA)(rA)(rG)(rU) | | |
| | | (rU)(rA)(rA)(rA)(rA)(rU)(rA)(rA)(rG)(rG)(rC) | | |
| | | (rU)(rA)(rG)(rU)(rC)(rC)(rG)(rU)(rU)(rA)(rU)(rC) | | |
| | | (rA)(rA)(rC)(rU)(rU)(rG)(rA)(rA)(rA](rA)(rA)(rG) | | |
| | | (rU)(rG)(rG)(rC)(rA)(rC)(rC)(rG)(rA)(rG)(rU)(rC) | | |
| | | (rG)(rG)(rU)(rG)(rC)#(mU)#(mU)#(mU) | | |
| | | (SEQ ID NO: 37) | | |

r indicates a ribonucleotide (2′-OH).
m indicates a 2′-O-methylated ribonucleotide (2′-OMe).
# indicates a PS linkage.
Underline indicates the 3 extra nucleotides on the crRNA and tracrRNA to enable 3 extra base pairs in the stem regions of ligated sgRNAs.

### Example III

### Synthesis Of Norbornene-Methanol Phosphoramidite

Synthesis of norbornene-methanol phosphoramidite was achieved essentially as previously reported [8]. Briefly, norbornene-methanol (1 eq) was dissolved in DCM to make a 2M solution, then added DIPEA (3 eq) and cooled in an ice bath. 2-Cyanoethyl N,N-diisopropylchlorophosphoramidite (1.5 eq) was added slowly to the reaction mixture and stirred for 45 min at room temperature. After confirming the reaction was complete by TLC, the reaction mixture was diluted with 50 mL of DCM. The organic layer was washed with aqueous saturated NaHCO₃ (25 mL×2) followed by saturated NaCl (25 mL×1) solution and dried over Na₂SO₄. The organic layer was then evaporated to dryness and purified by column chromatography using ethyl acetate-hexane as the eluent. The final purified norbornene-methanol phosphoramidite was characterized by ³¹P NMR (151.9 MHz, CDCl3, δ ppm: 147.47, 147.34, 147.32, 147.09). See, FIG. 2.

### Example IV

### Methyl-Tetrazine-NHS Ester Post-Synthesis Conjugation

Tetrazine functionalization of crRNAs was achieved post-synthetically via NHS-ester conjugation reaction between 3′-amine of crRNAs and methyl-tetrazine-NHS ester/methyl-tetrazine-PEG8-NHS ester. See, FIG. 23.

Briefly, methyl-tetrazine-NHS ester/methyl-tetrazine-PEG8-NHS ester dissolved in DMSO (35 equiv.) was added to a 1.5-2.0 mM RNA solution in 200 mM HEPES, pH 8.3 buffer, maintaining a 1:1 ratio of 200 mM HEPES, pH 8.3 buffer and DMSO to ensure optimal solubility of the tetrazine substrate. The mixture was incubated at 40° C. for 48-72h. The reaction mixture was desalted using Glen Pak desalting column to remove excess of the unreacted methyltetrazine-NHS ester/methyltetrazine-PEG8-NHS ester.

Further, RP-HPLC purification was done using C18 semipreparative column with a flow rate of 2.5 mL/min using linear gradient of 2-40% B (100% Acetonitrile). 0.1 M TEAA was used as buffer A. Reaction success was confirmed by HPLC-MS. See, Table 2.

### Example V

### sgRNA Formation By Tetrazine Ligation Of crRNA And tracrRNA

3′-tetrazine-modified crRNA and 5′-norbornene-modified tracrRNA (800 μM each) was mixed in 5 μL of 20 mM Tris-HCl, 200 mM NaCl, pH 7.4, and incubated at room temperature overnight (17-21 h).

### Example VI

### Denaturing Polyacrylamide Gel Electrophoresis

Ligation reaction mixtures were purified by 15% TBE-urea denaturing gel. Briefly, before loading onto gel plates, TBE-Urea 2× loading dye (Invitrogen) was added to the ligation reaction mixture in 1:1 ratio and heated at 80° C. to for 5 min to ensure complete denaturation. Gels were run at room temperature for 1.5 h at 150 V, stained with SYBR gold (1:10000) for 5 min and scanned in a gel imager.

### Example VII

### RNA Gel Extraction And Purification

After denaturing polyacrylamide gel electrophoresis in accordance with Example VI, the gel band containing tetrazine-ligated sgRNAs was excised, crushed, and immersed in extraction buffer (50 mM Tris-HCl, 25 mM NaCl, pH=7.5) for overnight shaking at 37° C.² The mixture was then centrifugally filtered in a Spin-X column to remove the gel and obtain oligonucleotide solution. The oligonucleotide solution was then desalted and washed using Amicon® Ultra 3K centrifugal filters per manufacturer instructions. Finally, the purified tetrazine-ligated sgRNAs were characterized by HPLC-MS. See, Table 1.

### Example VIII

### Cell Culture

HEK293T cells were obtained from ATCC. A HEK293T stable cell line expressing TLR1 (traffic light reporter 1) was cultured in Dulbecco's Modified Eagle Medium with 10% fetal bovine serum (Sigma) and 1% penicillin-streptomycin (Gibco). Cells were maintained in a humidified 37° C., 5% CO₂ incubator.

### Example IX

### Electroporation Of Mammalian Cells

HEK293T and HEK293T TLR1 cells were electroporated using the Neon transfection system (ThermoFisher) according to the manufacturer's protocol. SpyCas9 proteins were purchased from QB3-MacroLab, UC-Berkeley. Standard GAAA-tetraloop sgRNAs were purchased from Integrated DNA Technologies (IDT). Briefly, RNP complexes were made by mixing SpyCas9 and guide RNA in Buffer R (ThermoFisher) to a volume of 7-8 μl.

Cells were harvested, washed, and resuspended in the buffer R to the concentration of 100,000 cells per 5 μl. RNP complexes and 5 μl cells were then mixed and electroporated using a 10 μl Neon tip at the condition of 1150v, 20 ms, 2pulses. After electroporation, cells were plated in 24-well plates containing 500 μl DMEM with 10% FBS for 2-3 days until harvesting.

### Example X

### Flow Cytometry

After 2-3 days post electroporation, the TLR1 cells were trypsinized, collected by gentle centrifugation, and the pellet was resuspended in PBS containing 2% FBS. MACS-Quant VYB (Miltenyi Biotec) was used for flow cytometry. The live cells were first gated by forward scattering area (FSC-A) versus side scattering area (SSC-A). Then, the singlet cells were sorted using FSC-A versus FSC-H. Last, mCherry-expressing cells were detected using the yellow laser (561 nm) as excitation and 615/20 nm filter for emission. 10,000-20,000 events were collected and FlowJo was used for data analysis.

### Example XI

### Indel Analysis

After 2-3 days post electroporation, the genomic DNAs were extracted using DNeasy Blood and Tissue kit (Qiagen) or QuickExtract™ DNA Extraction Solution (Lucigen). 100-200 ng of genomic DNA was used for PCR amplification using the primers and NEBNext® Ultra™ II Q5® Master Mix. See, Table 3.

TABLE 3
Summary of representative PCR primers for Indel analysis.

| Locus | Forward primer (5′ to 3′) | Reverse primer (5′ to 3′) |
|---|---|---|
| CCR5 | AGAGCCAAGCTCTCCATCTA | GGAGAAGGACAATGTTGTAGGG |
| | (SEQ ID NO: 38) | (SEQ ID NO: 39) |
| HEK3 | CAATTACAACTTGACGGCTTTCT | GTCTCTGACCACTGCGATATG |
| | (SEQ ID NO: 40) | (SEQ ID NO: 41) |
| TRAC | AAGTTGGGGAGACCACTCCA | TGAGGCCTAGAAGAGCAGTA |
| | (SEQ ID NO: 42) | (SEQ ID NO: 43) |
| HPRT | TGATGCTCACCTCTCCCACA | CTGGCAAATGTGCCTCTCTA |
| | (SEQ ID NO: 44) | (SEQ ID NO: 45) |

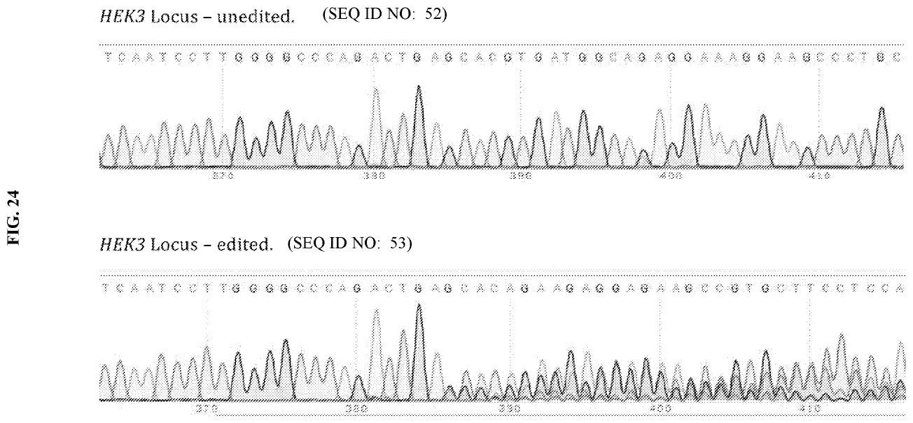

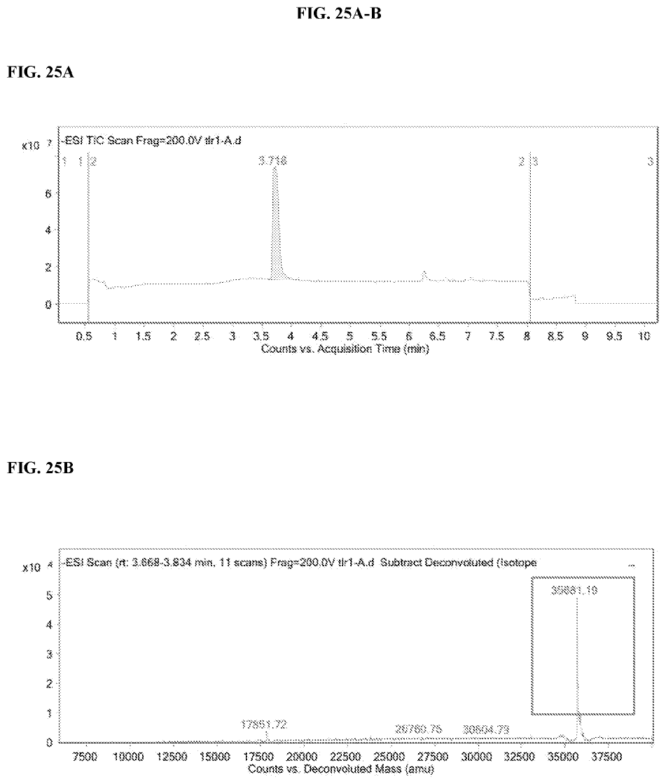

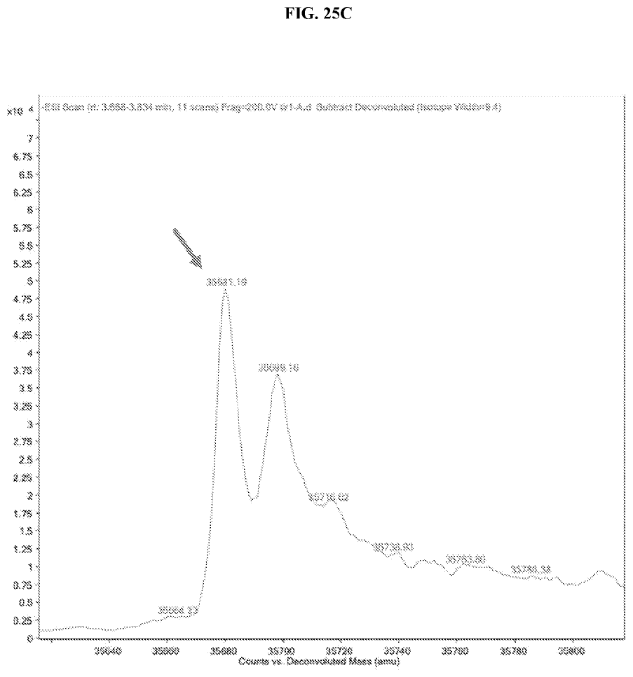

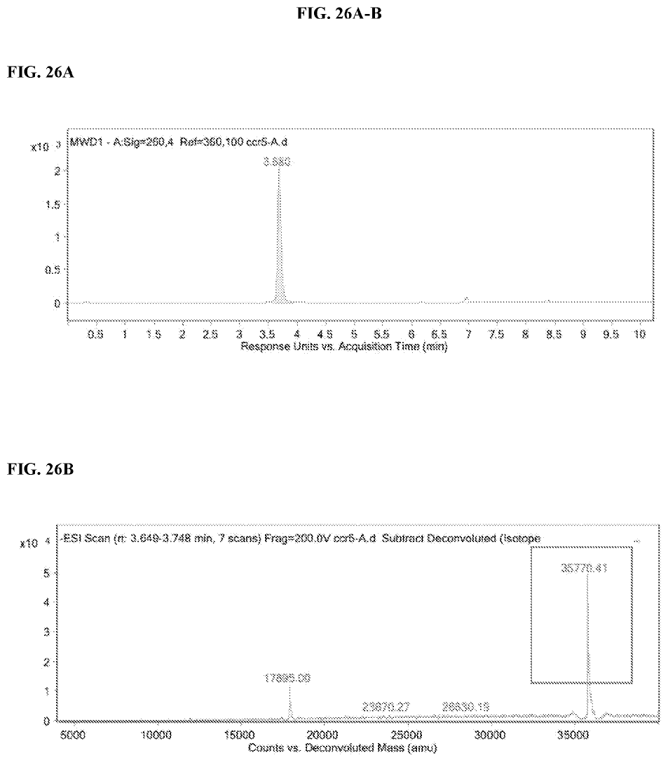

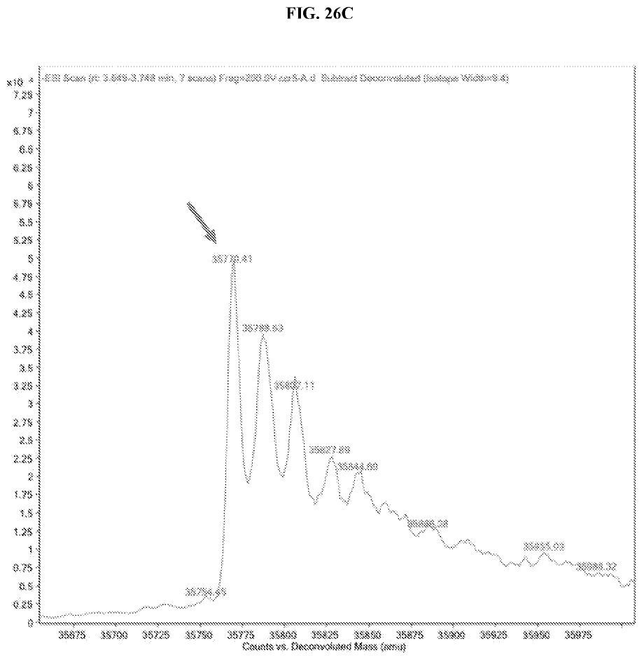

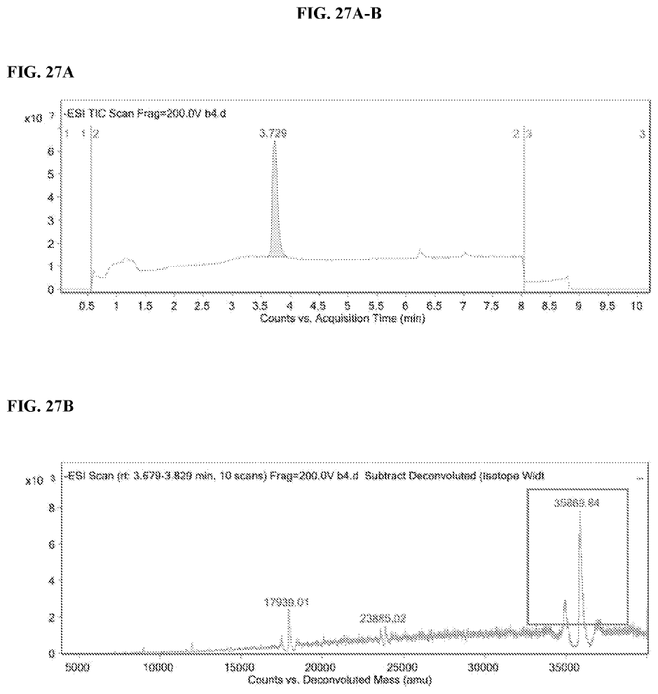

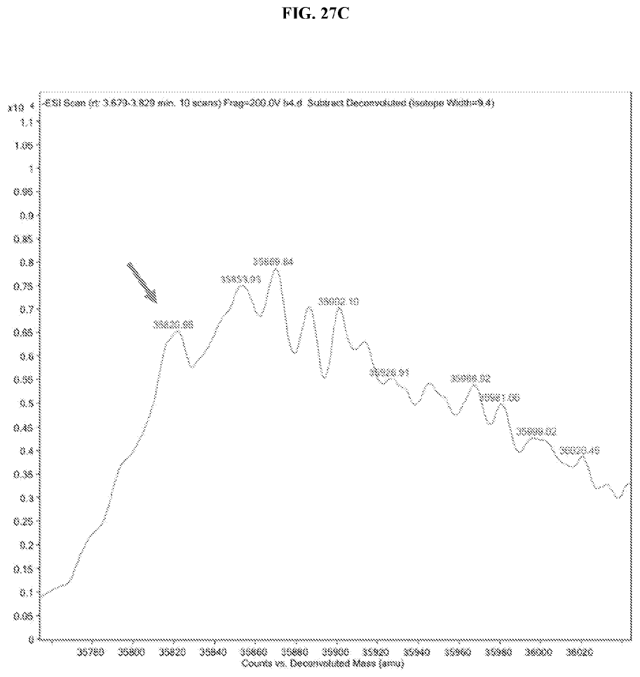

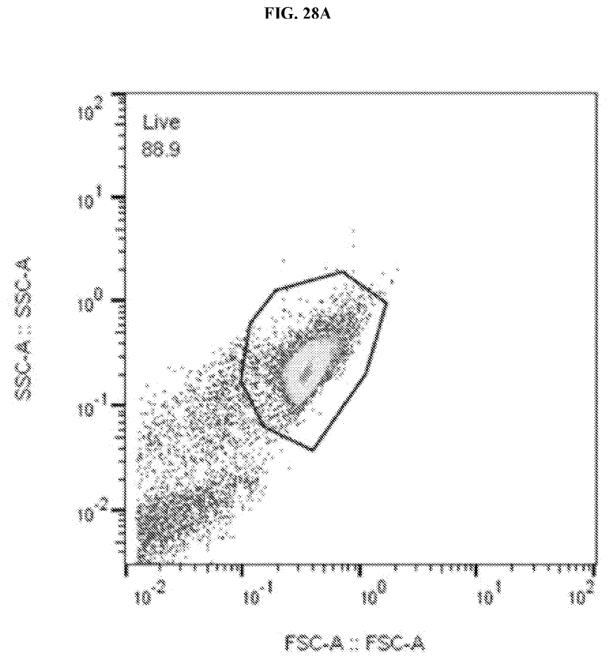

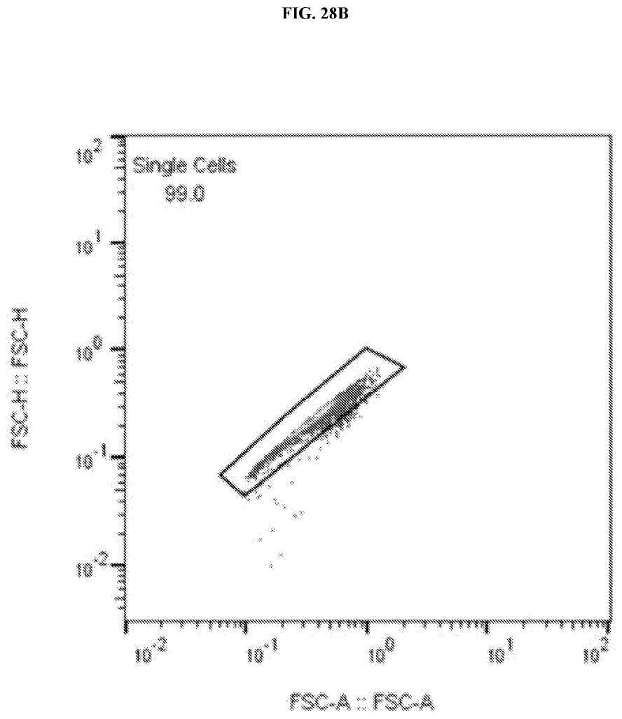

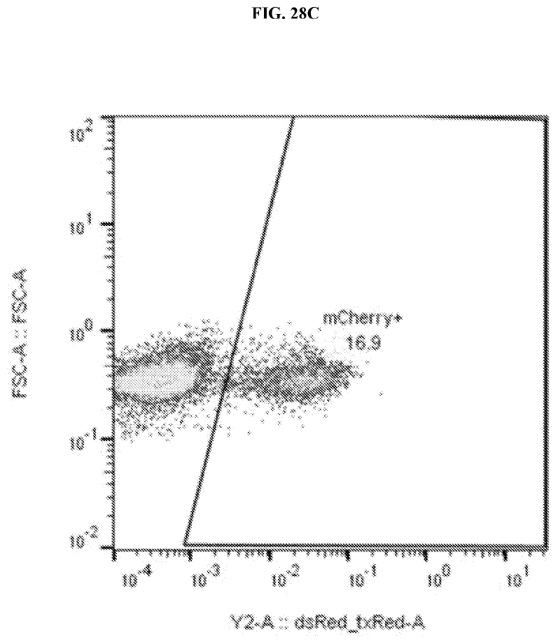

The PCR fragments were subjected to Sanger Sequencing. See, FIG. 24. and the sequencing files were analyzed using the ICE web tool to quantify Indel frequencies [9]. See, ice.synthego (dot) com.

### Example XII

### Electrophoretic Mobility Shift Assay (EMSA)

Briefly, Cas9 protein and guide RNA (either sgRNA or dgRNA) were mixed at molar ratio of 1:1 and incubated at room temperature for 30 minutes. Then RNP complexes were loaded and run on 2% agarose gel at 100 V in TBE running buffer for about 1 hour. EtBr was used as staining reagent.

### REFERENCES

1. Finn, J. D. et al. (2018) “A Single Administration of CRISPR/Cas9 Lipid Nanoparticles Achieves Robust and Persistent in vivo Genome Editing,” Cell Rep. 22 (9), 2227-2235.
2. Doudna, J. A. and Charpentier, E. (2014) “Genome Editing. The New Frontier of Genome Engineering with CRISPR-Cas9,” Science 346 (6213), Article number: 1258096.
3. Jinek, M. et al. (2012) “A Programmable Dual-RNA-Guided DNA Endonuclease in Adaptive Bacterial Immunity,” Science 337 (6096), 816-821.
4. Cong, L. et al. (2013) “Multiplex Genome Engineering Using CRISPR/Cas Systems,” Science (New York, N.Y.) 339 (6121), 819-823.
5. Esvelt, K. M. et al. (2013) “Orthogonal Cas9 Proteins for RNA-Guided Gene Regulation and Editing,” Nature Methods 10 (11), 1116-1121.
6. Fu, Y. et al. (2014) “Improving CRISPR-Cas Nuclease Specificity Using Truncated Guide RNAs,” Nat. Biotechnol. 32 (3), 279-284.
7. Edraki, A. et al. (2019) “A Compact, High-Accuracy Cas9 with a Dinucleotide Pam for in Vivo Genome Editing,” Mol. Cell 73 (4), 714-726.e714.
8. Urnov, F. D. (2018) “Genome Editing B.C. (before CRISPR): Lasting Lessons from the “Old Testament”,” The CRISPR Journal I (1), 34-46.
9. Jinek, M. et al. (2013) “RNA-Programmed Genome Editing in Human Cells,” eLife 2, e00471.
10. Mali, P. et al. (2013) “RNA-Guided Human Genome Engineering Via Cas9,” Science 339 (6121), 823-826.
11. Glass, Z. et al. (2018) “Engineering the Delivery System for CRISPR-Based Genome Editing,” Trends Biotechnol. 36 (2), 173-185.
12. Mir, A. et al. (2018) “Heavily and Fully Modified RNAs Guide Efficient Spycas9-Mediated Genome Editing,” Nat. Commun. 9 (1), 2641.
13. Yin, H. et al. (2017) “Structure-Guided Chemical Modification of Guide RNA Enables Potent Non-Viral in vivo Genome Editing,” Nat. Biotechnol. 35 (12), 1179-1187.
14. Rahdar, M. et al. (2015) “Synthetic CRISPR RNA-Cas9-Guided Genome Editing in Human Cells,” P.N.A.S. 112 (51), E7110-E7117.
15. Cromwell, C. R. et al. (2018) “Incorporation of Bridged Nucleic Acids into CRISPR RNAs Improves Cas9 Endonuclease Specificity,” Nat. Commun. 9 (1), 1448.
16. Yin, H. et al. (2018) “Partial DNA-Guided Cas9 Enables Genome Editing with Reduced Off-Target Activity,” Nat. Chem. Biol. 14 (3), 311-316.
17. Rueda, F. O. et al. (2017) “Mapping the Sugar Dependency for Rational Generation of a DNA-RNA Hybrid-Guided Cas9 Endonuclease,” Nat. Commun. 8 (1), 1610.
18. Hendel, A. et al. (2015) “Chemically Modified Guide RNAs Enhance CRISPR-Cas Genome Editing in Human Primary Cells,” Nat. Biotechnol. 33 (9), 985-989.
19. Ryan, D. E. et al. (2017) “Improving CRISPR-Cas Specificity with Chemical Modifications in Single-Guide RNAs,” Nucleic Acids Res. 46 (2), 792-803.
20. Basila, M. et al. (2017) “Minimal 2′-O-Methyl Phosphorothioate Linkage Modification Pattern of Synthetic Guide RNAs for Increased Stability and Efficient CRISPR-Cas9 Gene Editing Avoiding Cellular Toxicity,” PLOS One 12 (11), e0188593.
21 El-Sagheer, A. H. and Brown, T. (2010) “New Strategy for the Synthesis of Chemically Modified RNA Constructs Exemplified by Hairpin and Hammerhead Ribozymes,” P.N.A.S. 107 (35), 15329-15334.
22. Nishimasu, H. et al. (2014) “Crystal Structure of Cas9 in Complex with Guide RNA and Target DNA,” Cell 156 (5), 935-949.
23. Jiang, F. and Doudna, J. A. (2017) “CRISPR-Cas9 Structures and Mechanisms,” Annual Review of Biophysics 46 (1), 505-529.
24. Jinek, M. et al. (2014) “Structures of Cas9 Endonucleases Reveal RNA-Mediated Conformational Activation,” Science 343 (6176), Article number: 1247997.
25 Taemaitree, L. et al. (2019) “An Artificial Triazole Backbone Linkage Provides a Split-and-Click Strategy to Bioactive Chemically Modified CRISPR sgRNA,” Nat. Commun. 10 (1), 1610.
26 Gaetke, L. M. and Chow, C. K. (2003) “Copper Toxicity, Oxidative Stress, and Antioxidant Nutrients,” Toxicology 189 (1), 147-163.
27. Jewett, J. C. and Bertozzi, C. R. (2010) “Cu-Free Click Cycloaddition Reactions in Chemical Biology,” Chem. Soc. Rev. 39 (4), 1272-1279.
28. Neumann, S. et al. (2020) “The Cuaac: Principles, Homogeneous and Heterogeneous Catalysts, and Novel Developments and Applications,” Macromol. Rapid Commun. 41 (1), 1900359.
29. Huang, T. P. et al. (2021) “Precision Genome Editing Using Cytosine and Adenine Base Editors in Mammalian Cells,” Nat. Protoc. 16 (2), 1089-1128.
30. Yeo, N. C. et al. (2018) “An Enhanced CRISPR Repressor for Targeted Mammalian Gene Regulation,” Nature Methods 15 (8), 611-616.
31. Hilton, I. B. et al. (2015) “Epigenome Editing by a CRISPR/Cas9-Based Acetyltransferase Activates Genes from Promoters and Enhancers,” Nat. Biotechnol. 33 (5), 510-517.
32. Ji, Q. et al. (2014) “Engineered Zinc-Finger Transcription Factors Activate Oct4 (Pou5f1), sox2, klf4, c-Myc (Myc) and Mir302/367,” Nucleic Acids Res. 42 (10), 6158-6167.
33. Blackman, M. L. et al. (2008) “The Tetrazine Ligation: Fast Bioconjugation Based on Inverse-Electron-Demand Diels-Alder Reactivity,” J. Am. Chem. Soc. 130 (41), 13518-13519.
34. Devaraj, N. K. et al. (2008) “Tetrazine-Based Cycloadditions: Application to Pretargeted Live Cell Imaging,” Bioconjugate Chemistry 19 (12), 2297-2299.
35. Devaraj, N. K. et al. (2009) “Fast and Sensitive Pretargeted Labeling of Cancer Cells through a Tetrazine/Trans-Cyclooctene Cycloaddition,” Angew. Chem. Int. Ed. 48 (38), 7013-7016.
36. Knall, A.-C. and Slugovc, C. (2013) “Inverse Electron Demand Diels-Alder (Iedda)-Initiated Conjugation: A (High) Potential Click Chemistry Scheme,” Chem. Soc. Rev. 42 (12), 5131-5142.
37. Oliveira, B. L. et al. (2017) “Inverse Electron Demand Diels-Alder Reactions in Chemical Biology,” Chem. Soc. Rev. 46 (16), 4895-4950.
38. Schoch, J. et al. (2010) “Post-Synthetic Modification of DNA by Inverse-Electron-Demand Diels-Alder Reaction,” J. Am. Chem. Soc. 132 (26), 8846-8847.
39. Schoch, J. et al. (2011) “Inverse Electron-Demand Diels-Alder Reactions for the Selective and Efficient Labeling of RNA,” Chemical Communications 47 (46), 12536-12537.
40. Schoch, J. et al. (2012) “Site-Specific One-Pot Dual Labeling of DNA by Orthogonal Cycloaddition Chemistry,” Bioconjugate Chemistry 23 (7), 1382-1386.
41. Šečkutė, J. et al. (2013) “Rapid Oligonucleotide-Templated Fluorogenic Tetrazine Ligations,” Nucleic Acids Res. 41 (15), e148-e148.
42. Asare-Okai, P. N. et al. (2014) “Site-Specific Fluorescence Labelling of RNA Using Bio-Orthogonal Reaction of Trans-Cyclooctene and Tetrazine,” Chemical Communications 50 (58), 7844-7847.
43. Pyka, A. M. et al. (2014) “Diels-Alder Cycloadditions on Synthetic RNA in Mammalian Cells,” Bioconjugate Chemistry 25 (8), 1438-1443.
44. Domnick, C. et al. (2015) “Site-Specific Enzymatic Introduction of a Norbornene Modified Unnatural Base into RNA and Application in Post-Transcriptional Labeling,” Chemical Communications 51 (39), 8253-8256.
45. Holstein, J. M. and Rentmeister, A. (2016) “Current Covalent Modification Methods for Detecting RNA in Fixed and Living Cells,” Methods 98, 18-25.
46. Winz, M.-L. et al. (2018) “Site-Specific One-Pot Triple Click Labeling for DNA and RNA,” Chemical Communications 54 (83), 11781-11784.
47. Certo, M. T. et al. (2011) “Tracking Genome Engineering Outcome at Individual DNA Breakpoints,” Nature Methods 8, 671-676.
48. Conant, D. et al. (2022) “Inference of CRISPR Edits from Sanger Trace Data,” The CRISPR Journal 5 (1), 123-130.
49. Gasiunas, G. et al. (2012) “Cas9-Crrna Ribonucleoprotein Complex Mediates Specific DNA Cleavage for Adaptive Immunity in Bacteria,” Proc. Natl. Acad. Sci. U.S.A 109 (39), E2579-E2586.
50. Briner, A. E. et al. (2014) “Guide RNA Functional Modules Direct Cas9 Activity and Orthogonality,” Mol. Cell 56 (2), 333-339.
51. Chen, B. et al. (2013) “Dynamic Imaging of Genomic Loci in Living Human Cells by an Optimized CRISPR/Cas System,” Cell 155 (7), 1479-1491.
52. Grevet, J. D. et al. (2018) “Domain-Focused CRISPR Screen Identifies Hri as a Fetal Hemoglobin Regulator in Human Erythroid Cells,” Science 361 (6399), 285-290.

1. A single guide ribonucleic acid (sgRNA) comprising a crRNA domain, a tracrRNA domain and a linker, said linker comprising a tetrazine-derived moiety and a dienophile-derived moiety.

2. The sgRNA of claim 1, wherein said dienophile-derived moiety comprises a norbornene-conjugated nucleic acid.

3-4. (canceled)

5. The sgRNA of claim 1, wherein said linker is ligated between crRNA domain and tracrRNA domain.

6. The sgRNA of claim 1, wherein said tetrazine-derived moiety is ligated to a 3′ terminus of the crRNA or a 5′ terminus of the tracrRNA.

7. The sgRNA of claim 1, wherein said dienophile-derived moiety is ligated to a 5′ terminus of the tracrRNA or a 3′ terminus of the crRNA.

8-9. (canceled)

10. The sgRNA of claim 1, wherein said linker further comprises an octaethylene glycol (PEG8) moiety or a tetraethylene glycol (PEG4) moiety.

11-14. (canceled)

15. The sgRNA of claim 1, wherein said linker is selected from the group consisting of a tetrazine-derived long linker 2, a tetrazine-derived long linker 3, a tetrazine-derived long linker 4, a tetrazine-derived long linker 5 and a tetrazine-derived long linker 6.

16. A method, comprising:
a) providing;
i) a tracrRNA molecule comprising a 5′-alkene or 5′-alkyne dienophile moiety;
ii) a crRNA molecule comprising a 3′-tetrazine moiety; and
b) ligating said 5′-alkene or 5′-alkyne dienophile moiety and said 3′-tetrazine moiety to create an sgRNA comprising a linker having a dienophile-derived moiety and a tetrazine-derived moiety.

17. (canceled)

18. The method of claim 16, wherein said 5′-alkene or 5′-alkyne dienophile moiety is derived from a phosphoramidite selected from the group consisting of 5′-norbornene phosphoramidite, a 5′-bicyclo[6.1.0] nonyne phosphoramidite and a 5′-trans-cyclooctene phosphoramidite.

19-24. (canceled)

25. The method of claim 16, wherein said 3′-tetrazine moiety further comprises an oligoethylene glycol or polyethylene glycol moiety.

26-27. (canceled)

28. The method of claim 16, wherein said method is metal-free.

29. (canceled)

30. A method, comprising:
a) providing:
i) a single guide ribonucleic acid (sgRNA) comprising a crRNA domain, a tracrRNA domain and a linker, comprising a tetrazine-derived moiety and a dienophile-derived moiety;
ii) a Cas9 nuclease; and
iii) a target nucleic acid that is at least partially complementary to said sgRNA;
b) contacting said sgRNA with said Cas9 nuclease to create a Cas9 nuclease/sgRNA complex; and
c) hybridizing said Cas9 nuclease/sgRNA complex to said target nucleic acid, wherein the sequence of said target nucleic acid is edited.

31. The method of claim 30, wherein said Cas9 nuclease is an inactivated Cas9 nuclease (dCas9) or a Cas9 nickase (nCas9).

32. (canceled)

33. The method of claim 30, wherein said target nucleic acid is derived from a gene.

34. The method of claim 33, wherein said gene is selected from the group consisting of CCR5, HEK3, TRAC, and HPRT.

35. The method of claim 30, wherein said target nucleic acid is linked to a genetic disease or disorder.

36. (canceled)

37. The method of claim 30, wherein said linker is ligated between said crRNA domain and said tracrRNA domain.

38. The method of claim 30, wherein said tetrazine-derived moiety is ligated to a 3′-terminus of the crRNA domain.

39. The method of claim 30, wherein said norbornene moiety is ligated to a 5-terminus of the tracrRNA domain.

40-44. (canceled)

45. The method of claim 30, wherein said linker is selected from the group consisting of a tetrazine-derived long linker 2, a tetrazine-derived long linker 3, a tetrazine-derived long linker 4, a tetrazine-derived long linker 5 and a tetrazine-derived long linker 6.

46. (canceled)


SEQUENCE LISTING (65 sequences)

SEQ ID NO: 1 - RNA, 36 nt, synthetic construct
  source 1..36: mol_type = other RNA
  modified_base 1..3: mod_base = OTHER; note = The residues at these positions are 2-O-methylated ribonucleotides.
  modified_base 36: mod_base = OTHER; note = The residue at this position contains the linking molecule of -(Tetrazine)-(Norbornene)-
ggtgagctcttatttgcgtagttttagagctatgct

SEQ ID NO: 2 - RNA, 36 nt, synthetic construct; identical to SEQ ID NO: 1
  source 1..36: mol_type = other RNA
  modified_base 1..3: mod_base = OTHER; note = The residues at these positions are 2-O-methylated ribonucleotides
  modified_base 36: mod_base = OTHER; note = The residue at this position contains the linking molecule of -(PEG8-tetrazine)-(Norbornene)-

SEQ ID NO: 3 - RNA, 36 nt, synthetic construct; identical to SEQ ID NO: 1
  source 1..36: mol_type = other RNA
  modified_base 1..3: mod_base = OTHER; note = The residues at these positions are 2-O-methylated ribonucleotides
  modified_base 36: mod_base = OTHER; note = The residue at this position contains the linking molecule of -(Tetrazine)-(Norbornene)-
  modified_base 1..4: mod_base = OTHER; note = There exists a Phosphorothioate linkage between these residues

SEQ ID NO: 4 - RNA, 36 nt, synthetic construct
  source 1..36: mol_type = other RNA
  modified_base 36: mod_base = OTHER; note = The residue at this position contains the linking molecule of -(PEG8-tetrazine)-(Norbornene)-
  modified_base 1..4: mod_base = OTHER; note = There exists a Phosphorothioate linkage between these residues
  modified_base 1..3: mod_base = OTHER; note = The residues at these positions are 2-O-methylated ribonucleotides.
gcagcatagtgagcccagaagttttagagctatgct

SEQ ID NO: 5 - RNA, 36 nt, synthetic construct
  source 1..36: mol_type = other RNA
  modified_base 36: mod_base = OTHER; note = The residue at this position contains the linking molecule of -(PEG8-tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues
  modified_base 10: mod_base = OTHER; note = The residue in this position is thymine.
ggcccagactgagcacgtgagttttagagctatgct

SEQ ID NO: 6 - RNA, 36 nt, synthetic construct
  source 1..36: mol_type = other RNA
  modified_base 36: mod_base = OTHER; note = The residue at this position contains the linking molecule of -(PEG8-tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues
gagaatcaaaatcggtgaatgttttagagctatgct

SEQ ID NO: 7 - RNA, 36 nt, synthetic construct
  source 1..36: mol_type = other RNA
  modified_base 36: mod_base = OTHER; note = The residue at this position contains the linking molecule of -(PEG8-tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues
ggtcacttttaacacacccagttttagagctatgct

SEQ ID NO: 8 - RNA, 36 nt, synthetic construct; identical to SEQ ID NO: 7
  source 1..36: mol_type = other RNA
  modified_base 36: mod_base = OTHER; note = The residue at this position contains the linking molecule of -(PEG4)-(Tetrazine)-(Norbornene)-(PEG4)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 9 - RNA, 36 nt, synthetic construct; identical to SEQ ID NO: 7
  source 1..36: mol_type = other RNA
  modified_base 36: mod_base = OTHER; note = The residue at this position contains the linking molecule of -(PEG4)-(PEG8-Tetrazine)-(Norbornene)-(PEG4)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 10 - RNA, 39 nt, synthetic construct
  source 1..39: mol_type = other RNA
  modified_base 39: mod_base = OTHER; note = The residue at this position contains the linking molecule of -(Tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues
ggtcacttttaacacacccagttttagagctatgctacg

SEQ ID NO: 11 - RNA, 39 nt, synthetic construct; identical to SEQ ID NO: 10
  source 1..39: mol_type = other RNA
  modified_base 39: mod_base = OTHER; note = The residue at this position contains the linking molecule of )-(PEG8-Tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 12 - RNA, 39 nt, synthetic construct
  source 1..39: mol_type = other RNA
  modified_base 39: mod_base = OTHER; note = The residue at this position contains the linking molecule of -(Tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues
ggtgagctcttatttgcgtagttttagagctatgctacg

SEQ ID NO: 13 - RNA, 39 nt, synthetic construct
  source 1..39: mol_type = other RNA
  modified_base 39: mod_base = OTHER; note = The residue at this position contains the linking molecule of -(Tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues
gcagcatagtgagcccagaagttttagagctatgctacg

SEQ ID NO: 14 - RNA, 39 nt, synthetic construct
  source 1..39: mol_type = other RNA
  modified_base 39: mod_base = OTHER; note = The residue at this position contains the linking molecule of -(Tetrazine)-(Norbornene)-
  modified_base 1..3: mod_base = OTHER; note = The residues at these positions are 2-O-methylated ribonucleotides
  modified_base 1..4: mod_base = OTHER; note = There exists a Phosphorothioate linkage between these residues
gagaatcaaaatcggtgaatgttttagagctatgctacg

SEQ ID NO: 15 - RNA, 36 nt, synthetic construct; identical to SEQ ID NO: 1
  source 1..36: mol_type = other RNA
  modified_base 1..3: mod_base = OTHER; note = The residues at these positions are 2-O-methylated ribonucleotides
  modified_base 36: mod_base = OTHER; note = The residue at this position contains the molecule C3-NH2

SEQ ID NO: 16 - RNA, 36 nt, synthetic construct; identical to SEQ ID NO: 1
  source 1..36: mol_type = other RNA
  modified_base 36: mod_base = OTHER; note = The residue at this position contains the molecule tetrazine
  modified_base 1..3: mod_base = OTHER; note = The residues at these positions are 2-O-methylated ribonucleotides

SEQ ID NO: 17 - RNA, 36 nt, synthetic construct; identical to SEQ ID NO: 1
  source 1..36: mol_type = other RNA
  modified_base 36: mod_base = OTHER; note = The residue at this position contains the molecule -(PEG8-tetrazine)
  modified_base 1..3: mod_base = OTHER; note = The residues at these positions are 2-O-methylated ribonucleotides

SEQ ID NO: 18 - RNA, 36 nt, synthetic construct; identical to SEQ ID NO: 1
  source 1..36: mol_type = other RNA
  modified_base 36: mod_base = OTHER; note = The residue at this position contains the molecule -C3-NH2
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 19 - RNA, 36 nt, synthetic construct; identical to SEQ ID NO: 1
  source 1..36: mol_type = other RNA
  modified_base 36: mod_base = OTHER; note = The residue at this position contains the molecule -(PEG8-tetrazine)
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 20 - RNA, 36 nt, synthetic construct; identical to SEQ ID NO: 4
  source 1..36: mol_type = other RNA
  modified_base 36: mod_base = OTHER; note = The residue at this position contains the molecule -C3-NH2
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 21 - RNA, 36 nt, synthetic construct; identical to SEQ ID NO: 4
  source 1..36: mol_type = other RNA
  modified_base 36: mod_base = OTHER; note = The residue at this position contains the molecule -(PEG8-tetrazine)
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 22 - RNA, 36 nt, synthetic construct; identical to SEQ ID NO: 5
  source 1..36: mol_type = other RNA
  modified_base 36: mod_base = OTHER; note = The residue at this position contains the molecule -C3-NH2
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 23 - RNA, 36 nt, synthetic construct; identical to SEQ ID NO: 5
  source 1..36: mol_type = other RNA
  modified_base 36: mod_base = OTHER; note = The residue at this position contains the molecule -(PEG8-tetrazine)
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 24 - RNA, 36 nt, synthetic construct; identical to SEQ ID NO: 6
  source 1..36: mol_type = other RNA
  modified_base 36: mod_base = OTHER; note = The residue at this position contains the molecule C3-NH2
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 25 - RNA, 36 nt, synthetic construct; identical to SEQ ID NO: 6
  source 1..36: mol_type = other RNA
  modified_base 36: mod_base = OTHER; note = The residue at this position contains the molecule (PEG8-tetrazine)
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 26 - RNA, 36 nt, synthetic construct; identical to SEQ ID NO: 7
  source 1..36: mol_type = other RNA
  modified_base 36: mod_base = OTHER; note = The residue at this position contains the molecule C3-NH2
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 27 - RNA, 36 nt, synthetic construct; identical to SEQ ID NO: 7
  source 1..36: mol_type = other RNA
  modified_base 36: mod_base = OTHER; note = The residue at this position contains the molecule (PEG8-tetrazine)
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 28 - RNA, 36 nt, synthetic construct; identical to SEQ ID NO: 7
  source 1..36: mol_type = other RNA
  modified_base 36: mod_base = OTHER; note = The residue at this position contains the molecule (PEG4)-C3-NH2
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 29 - RNA, 36 nt, synthetic construct; identical to SEQ ID NO: 7
  source 1..36: mol_type = other RNA
  modified_base 036: mod_base = OTHER; note = The residue at this position contains the molecule (PEG4)-tetrazine
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 30 - RNA, 36 nt, synthetic construct; identical to SEQ ID NO: 7
  source 1..36: mol_type = other RNA
  modified_base 36: mod_base = OTHER; note = The residue at this position contains the molecule (PEG4)-(PEG8-tetrazine)
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 31 - RNA, 39 nt, synthetic construct; identical to SEQ ID NO: 10
  source 1..39: mol_type = other RNA
  modified_base 36: mod_base = OTHER; note = The residue at this position contains the molecule C3-NH2
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 32 - RNA, 38 nt, synthetic construct
  source 1..38: mol_type = other RNA
  modified_base 038: mod_base = OTHER; note = The residue at this position contains the molecule tetrazine
  modified_base 1..3: mod_base = OTHER; note = The residues at these positions are 2-O-methylated ribonucleotides
  modified_base 1..4: mod_base = OTHER; note = There exists a Phosphorothioate linkage between these residues
ggtcactttaacacacccagttttagagctatgctacg

SEQ ID NO: 33 - RNA, 39 nt, synthetic construct; identical to SEQ ID NO: 10
  source 1..39: mol_type = other RNA
  modified_base 039: mod_base = OTHER; note = The residue at this position contains the molecule (PEG8-tetrazine)
  modified_base 1..3: mod_base = OTHER; note = The residues at these positions are 2-O-methylated ribonucleotides
  modified_base 1..4: mod_base = OTHER; note = There exists a Phosphorothioate linkage between these residues

SEQ ID NO: 34 - RNA, 67 nt, synthetic construct
  source 1..67: mol_type = other RNA
  modified_base 1: mod_base = OTHER; note = The residue at this position contains the molecule Norbornene
  modified_base 65..67: mod_base = OTHER; note = The residues at these positions are 2-O-methylated ribonucleotides
agcatagcaagttaaaataaggctagtccgttatcaacttgaaaaagtggcaccgagtcggtgcttt

SEQ ID NO: 35 - RNA, 67 nt, synthetic construct; identical to SEQ ID NO: 34
  source 1..67: mol_type = other RNA
  modified_base 1: mod_base = OTHER; note = The residue at this position contains the molecule Norbornene
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 36 - RNA, 67 nt, synthetic construct; identical to SEQ ID NO: 34
  source 1..67: mol_type = other RNA
  modified_base 1: mod_base = OTHER; note = The residue at this position contains the molecule Norbornene-(PEG4)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 37 - RNA, 70 nt, synthetic construct
  source 1..70: mol_type = other RNA
  modified_base 1: mod_base = OTHER; note = The residue at this position contains the molecule Norbornene
  modified_base 68..70: mod_base = OTHER; note = The residues at these positions are 2-O-methylated ribonucleotides
  modified_base 67..70: mod_base = OTHER; note = There exists a Phosphorothioate linkage between these residues
cgtagcatagcaagttaaaataaggctagtccgttatcaacttgaaaaagtggcaccgagtcggtgcttt

SEQ ID NO: 38 - DNA, 20 bp, synthetic construct
  source 1..20: mol_type = other DNA
agagccaagctctccatcta

SEQ ID NO: 39 - DNA, 22 bp, synthetic construct
  source 1..22: mol_type = other DNA
ggagaaggacaatgttgtaggg

SEQ ID NO: 40 - DNA, 23 bp, synthetic construct
  source 1..23: mol_type = other DNA
caattacaacttgacggctttct

SEQ ID NO: 41 - DNA, 21 bp, synthetic construct
  source 1..21: mol_type = other DNA
gtctctgaccactgcgatatg

SEQ ID NO: 42 - DNA, 20 bp, synthetic construct
  source 1..20: mol_type = other DNA
aagttggggagaccactcca

SEQ ID NO: 43 - DNA, 20 bp, synthetic construct
  source 1..20: mol_type = other DNA
tgaggcctagaagagcagta

SEQ ID NO: 44 - DNA, 20 bp, synthetic construct
  source 1..20: mol_type = other DNA
tgatgctcacctctcccaca

SEQ ID NO: 45 - DNA, 20 bp, synthetic construct
  source 1..20: mol_type = other DNA
ctggcaaatgtgcctctcta

SEQ ID NO: 46 - RNA, 12 nt, synthetic construct
  source 1..12: mol_type = other RNA
gctagaaatagc

SEQ ID NO: 47 - RNA, 16 nt, synthetic construct
  source 1..16: mol_type = other RNA
gctatgctagcatagc

SEQ ID NO: 48 - RNA, 16 nt, synthetic construct
  source 1..16: mol_type = other RNA
gctatgctagcatagc

SEQ ID NO: 49 - RNA, 16 nt, synthetic construct
  source 1..16: mol_type = other RNA
gctatgctagcatagc

SEQ ID NO: 50 - RNA, 22 nt, synthetic construct
  source 1..22: mol_type = other RNA
gctatgctacgcgtagcatagc

SEQ ID NO: 51 - RNA, 22 nt, synthetic construct
  source 1..22: mol_type = other RNA
gctatgctacgcgtagcatagc

SEQ ID NO: 52 - RNA, 67 nt, synthetic construct; identical to SEQ ID NO: 34
  source 1..67: mol_type = other RNA
  modified_base 1: mod_base = OTHER; note = The residue at this position contains the linking molecule -(Tetrazine)-(Norbornene)-
  modified_base 65..67: mod_base = OTHER; note = The residues at these positions are 2-O-methylated ribonucleotides

SEQ ID NO: 53 - RNA, 67 nt, synthetic construct; identical to SEQ ID NO: 34
  source 1..67: mol_type = other RNA
  modified_base 1: mod_base = OTHER; note = The residue at this position contains the linking molecule -(PEG8-tetrazine)-(Norbornene)-
  modified_base 65..67: mod_base = OTHER; note = The residues at these positions are 2-O-methylated ribonucleotides

SEQ ID NO: 54 - RNA, 67 nt, synthetic construct; identical to SEQ ID NO: 34
  source 1..67: mol_type = other RNA
  modified_base 1: mod_base = OTHER; note = The residue at this position contains the linking molecule -(PEG8-tetrazine)-(Norbornene)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 55 - RNA, 67 nt, synthetic construct; identical to SEQ ID NO: 34
  source 1..67: mol_type = other RNA
  modified_base 1: mod_base = OTHER; note = The residue at this position contains the linking molecule -(PEG8-tetrazine)-(Norbornene)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 56 - RNA, 67 nt, synthetic construct; identical to SEQ ID NO: 34
  source 1..67: mol_type = other RNA
  modified_base 1: mod_base = OTHER; note = The residue at this position contains the linking molecule -(PEG8-tetrazine)-(Norbornene)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 57 - RNA, 67 nt, synthetic construct; identical to SEQ ID NO: 34
  source 1..67: mol_type = other RNA
  modified_base 65..67: mod_base = OTHER; note = The residue at this position contains the linking molecule -(PEG8-tetrazine)-(Norbornene)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 58 - RNA, 67 nt, synthetic construct; identical to SEQ ID NO: 34
  source 1..67: mol_type = other RNA
  modified_base 1: mod_base = OTHER; note = The residue at this position contains the linking molecule -(PEG8-tetrazine)-(Norbornene)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 59 - RNA, 67 nt, synthetic construct; identical to SEQ ID NO: 34
  source 1..67: mol_type = other RNA
  modified_base 1: mod_base = OTHER; note = The residue at this position contains the linking molecule -(PEG4)-(Tetrazine)-(Norbornene)-(PEG4)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 60 - RNA, 67 nt, synthetic construct; identical to SEQ ID NO: 34
  source 1..67: mol_type = other RNA
  modified_base 1: mod_base = OTHER; note = The residue at this position contains the linking molecule -(PEG4)-(PEG8-Tetrazine)-(Norbornene)-(PEG4)-
  modified_base 65..67: mod_base = OTHER; note = The residues at these positions are 2-O-methylated ribonucleotides
  modified_base 64..67: mod_base = OTHER; note = There exists a Phosphorothioate linkage between these residues

SEQ ID NO: 61 - RNA, 70 nt, synthetic construct; identical to SEQ ID NO: 37
  source 1..70: mol_type = other RNA
  modified_base 1: mod_base = OTHER; note = The residue at this position contains the linking molecule )-(Tetrazine)-(Norbornene
  modified_base 68..70: mod_base = OTHER; note = The residues at these positions are 2-O-methylated ribonucleotides
  modified_base 67..70: mod_base = OTHER; note = There exists a Phosphorothioate linkage between these residues

SEQ ID NO: 62 - RNA, 70 nt, synthetic construct; identical to SEQ ID NO: 37
  source 1..70: mol_type = other RNA
  modified_base 1: mod_base = OTHER; note = The residue at this position contains the linking molecule -(PEG8-Tetrazine)-(Norbornene)-
  modified_base 68..70: mod_base = OTHER; note = The residues at these positions are 2-O-methylated ribonucleotides
  modified_base 67..70: mod_base = OTHER; note = There exists a Phosphorothioate linkage between these residues

SEQ ID NO: 63 - RNA, 70 nt, synthetic construct; identical to SEQ ID NO: 37
  source 1..70: mol_type = other RNA
  modified_base 1: mod_base = OTHER; note = The residue at this position contains the linking molecule -(Tetrazine)-(Norbornene
  modified_base 68..70: mod_base = OTHER; note = The residues at these positions are 2-O-methylated ribonucleotides
  modified_base 67..70: mod_base = OTHER; note = There exists a Phosphorothioate linkage between these residues

SEQ ID NO: 64 - RNA, 70 nt, synthetic construct; identical to SEQ ID NO: 37
  source 1..70: mol_type = other RNA
  modified_base 1: mod_base = OTHER; note = The residue at this position contains the linking molecule )-(Tetrazine)-(Norbornene)-
  modified_base 68..70: mod_base = OTHER; note = The residues at these positions are 2-O-methylated ribonucleotides
  modified_base 67..70: mod_base = OTHER; note = There exists a Phosphorothioate linkage between these residues

SEQ ID NO: 65 - RNA, 70 nt, synthetic construct; identical to SEQ ID NO: 37
  source 1..70: mol_type = other RNA
  modified_base 1: mod_base = OTHER; note = The residue at this position contains the linking molecule (Tetrazine)-(Norbornene)-
  modified_base 68..70: mod_base = OTHER; note = The residues at these positions are 2-O-methylated ribonucleotides
  modified_base 67..70: mod_base = OTHER; note = There exists a Phosphorothioate linkage between these residues